(19) United States
(12) Patent Application Publication
(10) Pub. No.: US 2021/0317090 A1
(43) Pub. Date: Oct. 14, 2021

(54) AMINO ACID DEPLETION AGENTS AS ANTIPROLIFERATIVE AGENTS

(71) Applicants: University of Central Florida Research Foundation, Inc., Orlando, FL (US); Terrey Pines Institute for Molecular Studies, Port St. Lucie, FL (US)

(72) Inventors: Otto PHANSTIEL, IV, Oviedo, FL (US); Chelsea MASSARO, Apopka, FL (US); Jenna THOMAS, Winter Park, FL (US); Adel NEFZI, Port St. Lucie, FL (US)

(21) Appl. No.: 17/272,436

(22) PCT Filed: Aug. 30, 2019
(86) PCT No.: PCT/US2019/048972

§ 371 Date: Mar. 1, 2021

Related U.S. Application Data
(60) Provisional application No. 62/724,850, filed on Aug. 30, 2018.

(51) Int. Cl. C07D 241/04; A61K 45/06; A61K 31/337

(52) U.S. Cl. CPC C07D 241/04; A61K 31/337; A61K 45/06

(57) ABSTRACT

Novel compounds are described which decrease the intracellular levels of leucine and methionine. Treatment with these amino acid depletion agents affects many metabolic and life processes which rely upon methionine, leucine and their derivatives. Methionine depletion not only inhibits protein synthesis, but also polyamine biosynthesis and significantly reduces intracellular pools of the native polyamines, spermidine and spermine. Since methionine restriction has been shown to mimic caloric restriction in life extension studies across multiple species, these compounds are also expected to extend lifespan by limiting methionine supply.

### CROSS-REFERENCE TO RELATED APPLICATION

This application claims the benefit of United States Provisional Patent Application No. 62/724,850, filed Aug. 30, 2018, which is incorporated by reference herein in its entirety.

### STATEMENT REGARDING FEDERALLY SPONSORED RESEARCH OR DEVELOPMENT

This invention was made with government support under award number W81XWH-16-1-0370 awarded by the Army Medical Research and Material Command. The government has certain rights in this invention.

### BACKGROUND

Pancreatic cancer is expected to become the second leading cause of cancer related death by 2030, and existing medicines only extend life for 6-11 months, new medicines are desperately needed. While there are no papers detailing the intracellular depletion of amino acids by the disclosed structures herein, there are papers which mention structures similar to this class and a single report detailing a solid phase synthesis approach.

Methionine deprivation is a proven anticancer strategy and investigators have developed several methods to induce methionine depletion in cells and humans. In terms of cellular mechanism, methionine deficiency was found to cause a drastic decrease in protein translation via impaired start site recognition leading to growth arrest. Previous approaches generate intracellular methionine depletion centered on inhibiting its import into cells via the large amino acid transporter 1 (LAT-1). The LAT-1 complex on the surface of cells is a heterodimer of SLC3A2 and SLC7A5. LAT-1 imports hydrophobic amino acids such as methionine, leucine, and phenylalanine in exchange for intracellular glutamine stores. In short, this antiporter secretes glutamine and imports large hydrophobic amino acids. Current LAT-1 inhibitor designs are predicated upon phenylalanine/tyrosine (amino acid scaffolds). The prior idea was to present the cell surface receptor with a non-native amino acid motif with large bulky non-native side chain in hopes of competitively blocking the LAT-1 mediated uptake of native amino acids. Most of these prior agents have low potency and require mM levels to be effective. Other prior art infused patients with methioninase, an enzyme which degrades methionine to alpha-ketoacids, ammonia, and methanethiol. This agent effectively reduced plasma methionine levels to 50% of basal levels in a human breast cancer patient given a ten-hour infusion of 20,000 units of methioninase. This approach was also demonstrated in neuroblastomas. While the methioninase approach is effective, later work showed that mice treated with methioninase recover within 14 hours due to uptake of methionine from the diet. This led investigators to try dietary restrictions as an adjuvant therapy. Plasma methionine can be lowered to a <5 μM in mice with a combination of dietary restriction of methionine, homocysteine, and choline along with intraperitoneal injections of 1,000 U/kg L-methioninase and 25-50 mg/kg homocystine, each administered at 12-hour intervals. This later approach was well tolerated in mice and resulted in tumor stasis in 100% of treated animals within 4 days of treatment. This combination approach holds great promise for anticancer therapy, but the dietary restriction requirement may affect patient compliance and quality of life. For at least these reasons, a need exists for more efficient methods to deplete cells of methionine, especially methods where methionine import cannot circumvent the methionine depletion strategy.

### BRIEF SUMMARY

Various embodiments provide efficient methods to deplete cells of methionine, including methods where methionine import cannot circumvent the methionine depletion strategy. Various embodiments may obviate the need for dietary restrictions. Indeed, various embodiments which impact methionine and other amino acid levels like leucine (which is involved in mTOR signaling) offer a new approach to inhibit cell growth via amino acid restriction. As will be shown here, pancreatic cancer cells remain methionine-depleted even though their LAT-1 transporter is functional and sufficient methionine is present outside the cell.

Various embodiments relate to a compound having a structure selected from Formula A, Formula B, and Formula C,

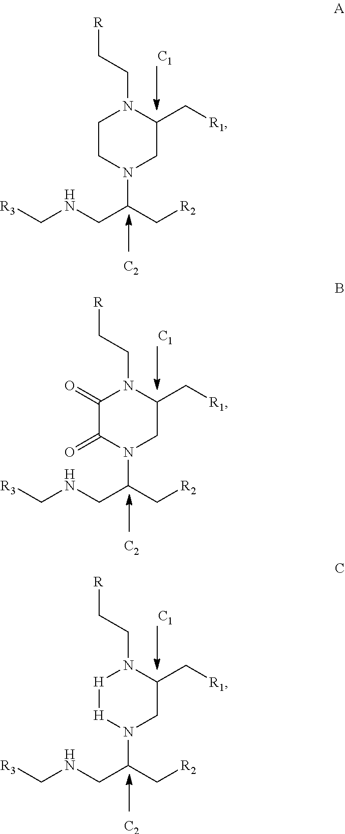

in which:
R may be selected from hydrogen, an aliphatic substituent, an alkylaryl substituent, a cycloalkyl substituent, an alkylcycloalkyl substituent and an aryl substituent,
R₁ may be selected from hydrogen, an aliphatic substituent, an alkylaryl substituent, a cycloalkyl substituent, an alkylcycloalkyl substituent, and an aryl substituent,
R₂ may be selected from hydrogen, an aliphatic substituent, an alkylaryl substituent, a cycloalkyl substituent, an alkylcycloalkyl substituent, and an aryl substituent,
R₃ may be selected from hydrogen, an aliphatic substituent, an alkylaryl substituent, a cycloalkyl substituent, an alkylcycloalkyl substituent, and an aryl substituent,
   C₁ may be a first chiral center. C₂ may be a second chiral center. The compound may have four stereoisomers, including an S,S stereoisomer, an R,R stereoisomer, an S,R stereoisomer, and an R,S stereoisomer. According to various embodiments, the compound may be any of the four stereoisomers. According to various embodiments, the compound may be the S,S stereoisomer. According to various embodiments, the compound may be the R,R stereoisomer.

According to various embodiments, R may be selected from methyl, ethyl, 1-propyl, 2-propyl, 1-butyl, isobutyl, sec-butyl, and tert-butyl. R may also be selected from cyclohexyl, phenyl, 4-fluorophenyl, benzyl, 4-fluorobenzyl, 2-pyridyl, and 3-pyridyl. R may also be selected from 1,1′-diphenylmethyl, or 3-(trifluoromethyl)phenyl, and bis-3,5-(trifluoromethyl)phenyl. R may also be selected from CH(CH₃)₂ and CH₂CH(CH₃)₂.

According to various embodiments, R₁ may be selected from 4-fluorophenyl, phenyl, 1-propyl, 2-propyl, isobutyl, sec-butyl, tert-butyl, 4-fluorobenzyl, and benzyl. R₁ may be cyclohexyl.

According to various embodiments, R₂ may be selected from hydrogen, methyl, ethyl, 1-propyl, 2-propyl, isobutyl, sec-butyl, tert-butyl, phenyl, benzyl, 4-hydroxyphenyl, 4-methoxyphenyl, 4-fluorophenyl, and cyclohexyl.

According to various embodiments, R₃ may be selected from hydrogen, cyclohexyl, 4-fluorophenyl, phenyl, 4-fluorobenzyl, and benzyl. R₃ may also be selected from methyl, ethyl, 1-propyl, 2-propyl, butyl, sec-butyl, isobutyl, cyclohexyl and cyclohexylmethyl. R₃ may also be selected from cyclopentyl and 4-methylphenyl. R₃ may also be selected from 4-fluorophenyl, phenyl and cyclohexyl.

According to various embodiments, the structure of the compound may be Formula A; R may be isopropyl; R₁ may be isopropyl; R₂ may be cyclohexyl; R₃ may be phenyl, and both C₁ and C₂ may be in the S isomer configuration.

According to various embodiments, the structure of the compound may be Formula A; R may be tert-butyl; R₁ may be selected from phenyl or 4-fluorophenyl; R₂ may be selected from cyclohexyl, phenyl or 4-fluorophenyl; R₃ may be selected from phenyl or 4-fluorophenyl; and both C₁ and C₂ may be in the S isomer configuration.

According to various embodiments, the structure of the compound may be Formula A; R may be isopropyl, R₁ may be isopropyl, R₂ may be cyclohexyl; R₃ may be selected from phenyl or 4-fluorophenyl; and both C₁ and C₂ may be in the R isomer configuration.

According to various embodiments, the structure of the compound may be Formula A; R may be t-butyl; R₁ may be phenyl or 4-fluorophenyl; R₂ may be selected from cyclohexyl, phenyl or 4-fluorophenyl; R₃ may be selected from phenyl or 4-fluorophenyl; and both C₁ and C₂ may be in the R isomer configuration.

According to various embodiments, the structure of the compound may be Formula A; R may be isopropyl; R₁ may be isopropyl; R₂ may be cyclohexyl; R₃ may be 4-fluorophenyl; and both C₁ and C₂ may be in the S isomer configuration.

According to various embodiments, the structure of the compound may generally be Formula A (or more specifically the structure illustrated below); R may be 2-propyl, R₁ may be 2-propyl, R₂ may be 2-propyl, R₃ may be 2-propyl; and both C₁ and C₂ may be in the S isomer configuration, as illustrated in the structure below.

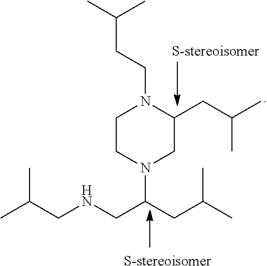

Various embodiments relate to a method that includes administering an effective dosage of the compound according to the various embodiments to a patient to treat a cancer. According to various embodiments, the cancer may be selected from pancreatic cancer, breast cancer, colorectal cancer, prostate cancer, lung cancer, and melanoma.

Various embodiments relate to a method that includes administering an effective dosage of the compound according to any of the various embodiments to a patient to treat a parasitic disease, which relies on amino acid supply from their host for survival. According to various embodiments, the parasitic disease may be selected from malaria, tuberculosis, Leishmania and Chagas disease.

Various embodiments relate to a method that includes administering an effective dosage of the compound according to the various embodiments to function as a depletion agent of one selected from leucine and methionine.

Various embodiments relate to a method that includes administering an effective dosage of the compound according to the various embodiments to function as a therapeutic in cells selected from mammalian cells and bacterial cells.

Various embodiments relate to a therapeutic composition that may include one or more of the compounds according to the various embodiments, and at least one antiproliferative agent. According to various embodiments, the antiproliferative agent may be selected from gemcitabine, difluoromethylornithine, a taxane derivative, and antifolate drugs. According to various embodiments, the taxane derivative may be taxol.

Various embodiments relate to methods that include administering an effective dosage of the compound according to the various embodiments to function as a therapeutic which lowers intracellular methionine pools. Various embodiments relate to methods of administering an effective dosage of the compound according to the various embodiments to function as a therapeutic which lowers intracellular methionine pools to provide extended life span.

Various embodiments relate to a method for synthesizing a compound according to the various embodiments described herein. The method including preparing a triamide scaffold; preparing a chiral triamine by reducing the triamide scaffold; preparing a diamine scaffold by regioselectively N-benzoylating the triamine scaffold; optionally regiospecifically cyclizing the diamine scaffold to prepare a cyclized scaffold; and reducing the diamine scaffold or the cyclized scaffold to form the compound. According to various embodiments of the method, preparing the triamide scaffold may include coupling a plurality of peptides. According to various embodiments of the method, preparing the triamide scaffold comprises: coupling an N-acylated amino acid to either D- or L-cyclohexylalanine methyl ester hydrochloride to produce a diamidoester, and converting the diamidoester to the triamide scaffold using ammonia gas.

These and other features, aspects, and advantages of various embodiments will become better understood with reference to the following description, figures, and claims.

### BRIEF DESCRIPTION OF THE FIGURES

Many aspects of this disclosure can be better understood with reference to the following figures, in which:

FIG. 1: is an example according to various embodiments, illustrating polyamine metabolism and LAT-1 (also known as SLC7A5);

FIG. 2: illustrates chemical structures of prior art inhibitors of polyamine metabolism (1-3), polyamine import (4) and LAT-1 (5-8);

FIG. 3: is an example according to various embodiments, illustrating lead architecture (A) identified from molecular library screening, top methionine depletion hits 9 and 10, and 11 (Ant44, a fluorescent cytotoxic polyamine);

FIG. 4A: is an example according to various embodiments, illustrating the inability of Compound 9 (1666.177) to prevent Spd from rescuing DFMO-treated CHO K1 cells;

FIG. 4B: is an example according to various embodiments, illustrating the inability of Compound 10 (1666.255) to prevent Spd from rescuing DFMO-treated CHO K1 cells;

FIG. 5: is an example according to various embodiments, illustrating potentiation of Ant-44 toxicity by compounds 9 and 10 in Chinese hamster ovary (CHO K1) cells;

FIG. 6: is an example according to various embodiments, illustrating the ability of compounds 9 (1666.177) and 10 (1666.177) to potentiate the toxicity of Ant-44 in L3.6pl human pancreatic cancer cells;

FIG. 7: is an example according to various embodiments, illustrating how both single and combination therapies in L3.6pl cells with Ant-44 and compound 10 (1666.255) affect intracellular polyamine pools and Ant44 levels after 72 h incubation;

FIG. 8: is an example according to various embodiments, illustrating reduced intracellular polyamine levels (expressed as nmoles polyamine/mg protein) in L3.6pl cells dosed with increasing concentrations of compound 10 (1666.255) after cells were incubated for 72 h at 37° C.;

FIG. 9A: is an example according to various embodiments, illustrating L3.6pl cells dosed with compound 10 at 0 μM;

FIG. 9B: is an example according to various embodiments, illustrating L3.6pl cells dosed with compound 10 at 2 μM;

FIG. 9C: is an example according to various embodiments, illustrating L3.6pl cells dosed with compound 10 at 5 μM;

FIG. 9D: is an example according to various embodiments, illustrating L3.6pl cells dosed with compound 10 at 7 μM;

FIG. 10A: is an example according to various embodiments, illustrating the inability of native polyamine putrescine (Put) to rescue L3.6pl cells treated with compound 10 at 1 μM and 5 μM;

FIG. 10B: is an example according to various embodiments, illustrating the inability of native polyamine spermidine (Spd) to rescue L3.6pl cells treated with compound 10 at 1 μM and 5 μM;

FIG. 10C: is an example according to various embodiments, illustrating inability of native polyamine spermine (Spm) to rescue L3.6pl cells treated with compound 10 at 1 μM and 5 μM;

FIG. 10D: is an example according to various embodiments, illustrating dose dependent decrease in 3H-Leucine uptake (as measured in counts per minute (CPM)) observed in the presence of increasing concentration of the known LAT-1 inhibitor JPH-203;

FIG. 10E: is an example according to various embodiments, illustrating results obtained for a Leu uptake inhibition experiment illustrating partial inhibition of Leucine import by compound 10;

FIG. 10F: is an example according to various embodiments, illustrating results obtained for a Leucine efflux experiment with LAT-1 inhibitor JPH-203;

FIG. 10G: is an example according to various embodiments, illustrating results obtained for a two minute Leucine efflux experiment with compound 10;

FIG. 10H: is an example according to various embodiments, illustrating results obtained for a thirty minute Leucine efflux experiment with compound 10;

FIG. 10I: is an example according to various embodiments, illustrating results with compound 10 and its effect on intracellular levels of polyamine metabolites;

FIG. 11: is an example according to various embodiments, illustrating an enlargement of Scheme 1; and

FIG. 12: is an example according to various embodiments, illustrating an enlargement of Scheme 2.

It should be understood that the various embodiments are not limited to the examples illustrated in the figures.

### DETAILED DESCRIPTION

Various embodiments may be understood more readily by reference to the following detailed description. Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

Unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

The present disclosure may be understood more readily by reference to the following detailed description of preferred embodiments of the disclosure as well as to the examples included therein. All numeric values are herein assumed to be modified by the term “about,” whether or not explicitly indicated. The term “about” generally refers to a range of numbers that one skilled in the art would consider equivalent to the recited value (i.e., having the same function or result). In many instances, the term “about” may include numbers that are rounded to the nearest significant figure. Generally, as used herein, the terms “about” and “approximately” refer to values that are ±10% of the stated value.

As used herein, the terms “administering” or “administration” of a compound or agent as described herein to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the term “analog” refers to a compound having a structure similar to that of another compound but differing from the other compound with respect to a certain component or substituent. The compound may differ in one or more atoms, functional groups, or substructures, which may be replaced with other atoms, groups, or substructures. In one aspect, such structures possess at least the same or a similar therapeutic efficacy.

The term “cancer” as used herein refers to a physiological condition in mammals that is typically characterized by unregulated cell growth. Exemplary cancers include, but are not limited to carcinoma, lymphoma, blastoma, sarcoma, and leukemia. More particularly, examples of such cancers include lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, neuroblastoma, spinal axis tumors, glioma, meningioma, and pituitary adenoma.

As used herein, the terms “co-administered, “co-administering,” or “concurrent administration”, when used, for example with respect to administration of a conjunctive agent along with administration of a composition as described herein refers to administration of an anti-metastatic agent as described herein and a conjunctive agent such that both can simultaneously achieve a physiological effect. The two agents, however, need not be administered together. In certain embodiments, administration of one agent can precede administration of the other, however, such co-administering typically results in both agents being simultaneously present in the body (e.g. in the plasma) of the subject.

As used herein, “derivative” refers to a compound derived or obtained from another and containing essential elements of the parent compound. In one aspect, such a derivative possesses at least the same or similar therapeutic efficacy as the parent compound.

As used herein, the terms “disease,” “disorder,” or “complication” refers to any deviation from a normal state in a subject. In preferred embodiments, the methods and compositions of the present invention are useful in the diagnosis and treatment of diseases characterized at least in part by cell proliferation and/or differentiation where control of methionine, leucine, or polyamine levels are required.

As used herein, by the term “effective amount,” “amount effective,” “therapeutically effective amount,” or the like, it is meant an amount effective at dosages and for periods of time necessary to achieve the desired result.

As used herein, the term “metastases” or “metastatic” refers to a secondary tumor that grows separately elsewhere in the body from the primary tumor and has arisen from detached, transported cells, wherein the primary tumor is a solid tumor. The primary tumor, as used herein, refers to a tumor that originated in the location or organ in which it is present and did not metastasize to that location from another location.

As used herein, term “pharmaceutically acceptable salt” is intended to include art-recognized pharmaceutically acceptable salts. These non-toxic salts are usually hydrolyzed under physiological conditions and include organic and inorganic acids and bases. Examples of salts include sodium, potassium, calcium, ammonium, copper, and aluminum as well as primary, secondary, and tertiary amines, polyamines, basic ion exchange resins, purines, piperazine, and the like. The term is further intended to include esters of lower hydrocarbon groups, such as methyl, ethyl, and propyl.

As used herein, the terms “composition” or “pharmaceutical composition” comprises one or more of the compounds described herein as active ingredient(s), or a pharmaceutically acceptable salt(s) thereof, and may also contain a pharmaceutically acceptable carrier and optionally other therapeutic ingredients. The compositions include compositions suitable for oral, rectal, ophthalmic, pulmonary, nasal, dermal, topical, parenteral (including subcutaneous, intramuscular and intravenous) or inhalation administration. The most suitable route in any particular case will depend on the nature and severity of the conditions being treated and the nature of the active ingredient(s). The compositions may be presented in unit dosage form and prepared by any of the methods well-known in the art of pharmacy. Dosage regimes may be adjusted for the purpose to improving the therapeutic response. For example, several divided dosages may be administered daily or the dose may be proportionally reduced over time. A person skilled in the art normally may determine the effective dosage amount and the appropriate regime.

As used herein, the term “preventing” means causing the clinical symptoms of a disorder or disease state, e.g., cancer, not to develop, e.g., inhibiting the onset of disease, in a subject that may be exposed to or predisposed to the disease state, but does not yet experience or display symptoms of the disease state.

As used herein, the term “prodrug” refers to a compound that is converted to a therapeutically active compound after administration, and the term should be interpreted as broadly herein as is generally understood in the art. Generally, but not necessarily, a prodrug is inactive or less active than the therapeutically active compound to which it is converted. For example, a methyl ester can be converted to a free carboxylic acid in vivo via the action of non-specific serum esterases.

As used herein, the term “stereoisomer” refers to a compound which has the identical chemical constitution but differs with regard to the arrangement of the atoms or groups in space.

As used herein, the term “subject” refers to any animal (e.g., a mammal), including, but not limited to, humans, which may be the recipient of a particular treatment. The term is intended to include living organisms susceptible to conditions or diseases caused or contributed to by unrestrained cell proliferation and/or differentiation where control of polyamine transport is required. Examples of subjects include, but are not limited to, humans, dogs, cats, horses, cows, goats, sheep, and mice. As used herein, the terms “treating” or “treatment” refer to both therapeutic treatment and prophylactic or preventative measures, wherein the object is to prevent or slow down (lessen) the targeted pathologic condition or disorder.

### Pharmaceutical Compositions

The compositions described herein may comprise an anti-metastatic agent as described herein. In one embodiment, there are provided pharmaceutical compositions comprising a compound of formula (I) above, or an analog, a derivative, a prodrug, a stereoisomer, or a pharmaceutically acceptable salt thereof, which can be administered to a patient to achieve a therapeutic effect, e.g., inhibit polyamine transport activity in the cells of a subject. In a particular embodiment, the pharmaceutical compound comprises a compound as described herein, or an analog, a derivative, a prodrug, a stereoisomer, or a pharmaceutically acceptable salt thereof. The compositions can be administered alone or in combination with at least one other agent, such as stabilizing compound, which can be administered in any sterile, biocompatible pharmaceutical carrier, including, but not limited to, saline, buffered saline, dextrose, and water. The compositions can be administered to a patient alone, or in combination with other agents, drugs or hormones, such as anti-cancer agents.

In addition to the active ingredients, these pharmaceutical compositions can contain suitable pharmaceutically acceptable carriers comprising excipients and auxiliaries, which facilitate processing of the active compounds into preparations which can be used pharmaceutically. Pharmaceutical compositions of the invention can be administered by any number of routes including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethylcellulose, or sodium carboxymethylcellulose; gums including arabic and tragacanth; and proteins such as gelatin and collagen. If desired, disintegrating or solubilizing agents can be added, such as the cross-linked polyvinyl pyrrolidone, agar, alginic acid, or a salt thereof, such as sodium alginate.

Dragee cores can be used in conjunction with suitable coatings, such as concentrated sugar solutions, which also can contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification or to characterize the quantity of active compound, i.e., dosage. Pharmaceutical preparations which will can be used orally include push fit capsules made of gelatin, as well as soft, sealed capsules made of gelatin and a coating, such as glycerol or sorbitol. Push fit capsules can contain active ingredients mixed with a filler or binders, such as lactose or starches, lubricants, such as talc or magnesium stearate, and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers also can be used for delivery. Optionally, the suspension also may contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee making, levigating, emulsifying, encapsulating, entrapping, or lyophilizing processes. The pharmaceutical composition can be provided as a salt and can be formed with many acids, including but not limited to, hydrochloric, sulfuric, acetic, lactic, tartaric, malic, succinic, etc. Alternatively, salts can be formed with many amine motifs such as primary, secondary and tertiary amines or even the native polyamines themselves. Salts tend to be more soluble in aqueous or other protonic solvents than are the corresponding free base or free acid forms.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent to a particular site in an animal, such as a human. Preferably, the lipid composition of the liposome is capable of targeting to a specific organ of an animal, such as the lung, liver, spleen, pancreas, heart brain, lymph nodes, and skin.

A liposome useful in the present invention comprises a lipid composition that is capable of fusing with the plasma membrane of the targeted cell to deliver its contents to the cell. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

### Determination of a Therapeutically Effective Dose

The determination of a therapeutically effective dose is well within the capability of those skilled in the art. A therapeutically effective dose refers to that amount of active ingredient which causes cytotoxicity to cancer cells and/or reduced metastatic behavior in a subject. Alternatively, a therapeutically effective dose may be determined by measuring blood plasma levels of the key molecules (methionine, leucine or polyamines) or their metabolites in response to drug dosage.

For any compound, the therapeutically effective dose can be estimated initially either in cell culture assays or in animal models, usually mice, rabbits, dogs, or pigs. The animal model also can be used to determine the appropriate concentration range and route of administration. Such information can then be used to determine useful doses and routes for administration in humans.

Therapeutic efficacy and toxicity, e.g., ED₅₀ (the dose therapeutically effective in 50% of the population) and LD₅₀ (the dose lethal to 50% of the population), can be determined by standard pharmaceutical procedures in cell cultures or experimental animals. The dose ratio of toxic to therapeutic effects is the therapeutic index, and it can be expressed as the ratio, LD₅₀/ED₅₀.

Pharmaceutical compositions which exhibit large therapeutic indices are preferred. The data obtained from cell culture assays and animal studies may be used in formulating a range of dosage for human use. The dosage contained in such compositions is preferably within a range of circulating concentrations that include the ED₅₀ with little or no toxicity. The dosage varies within this range depending upon the dosage form employed, sensitivity of the patient, and the route of administration. The toxicity of the present compounds of this invention can be further modulated by terminal N-alkylation. For example, polyamine compounds containing N-methyl groups are most stable to amine oxidases and are less toxic. These insights can be applied to the other compounds described herein. For example, tertiary amine systems should be stable to amine oxidases.

The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of container and labeled for treatment of an indicated condition. Such labeling would include amount, frequency, and method of administration.

Normal dosage amounts can vary from 0.1 to 100,000 micrograms, up to a total dose of about 1 g, depending upon the route of administration and duration of therapy. Guidance as to particular dosages and methods of delivery is provided in the literature and generally available to practitioners in the art. Those skilled in the art will employ different formulations for nucleotides than for proteins or their inhibitors.

In any of the embodiments described above, any of the pharmaceutical compositions of the invention can be administered in combination with other appropriate therapeutic agents. Selection of the appropriate agents for use in combination therapy can be made by one of ordinary skill in the art, according to conventional pharmaceutical principles. The combination of therapeutic agents can act synergistically to effect the treatment or prevention of the various disorders described above. Using this approach, one may be able to achieve therapeutic efficacy with lower dosages of each agent, thus reducing the potential for adverse side effects. Any of the therapeutic methods described above can be applied to any subject in need of such therapy, including, for example, mammals such as dogs, cats, cows, horses, rabbits, sheep, monkeys, and most preferably, humans.

### Applications

The compositions and methods described herein may be useful for the treatment and/or prevention of a cancer. In one embodiment, the methods and compositions may be utilized for the treatment of a metastatic cancer. It is appreciated that the cancer being treated may already have metastasized or is potentially metastatic. The cancer may comprise non-solid tumors, e.g., leukemia, multiple myeloma, hematologic malignancies or lymphoma. In another embodiment, the cancer is characterized by solid tumors and their potential or actual metastases including, but not limited to, melanoma; non-small cell lung cancer; glioma; hepatocellular (liver) carcinoma; glioblastoma; carcinoma and tumors of the thyroid, bile duct, bone, gastric, brain/CNS, head and neck; and hepatic, stomach, prostrate, breast, renal, testicular, ovarian, skin, cervical, lung, muscle, neuronal, esophageal, bladder, lung, uterine, vulval, endometrial, kidney, colorectal, pancreatic, pleural/peritoneal membranes, salivary gland, and epidermoid.

There are many other applications for methionine depletion agents beyond those described herein including life extension, including, for example, increased longevity, or as novel antibiotics. While caloric restricted diets have been shown to extend lifespan, methionine restricted diets can replace caloric restricted diets for extending the lifespan of animals and presumably humans. In another example, the tuberculosis causing organism (e.g., Mycobacterium tuberculosis) is very sensitive to methionine depletion and new therapies which starve these bacteria of methionine can be effective therapeutics.

### Conjunctive Delivery

In accordance with another aspect, there is provided a method for preventing or treating a cancer in a subject. The method comprises (a) administering to a subject a composition comprising a compound according to formula (I) in an amount effective to inhibit metastatic activity in the subject; and (b) administering at least one of radiation or a cytotoxic chemotherapeutic agent to the subject in an amount effective to induce a cytotoxic effect in cancer cells of the subject. The administering steps (a) and (b) may comprise inserting a delivery mechanism into the subject. The delivery mechanism comprises a structure insertable into the subject through which the composition can be delivered and an actuating mechanism for directing the composition into the subject. The use of such a delivery mechanism may be applied to any other embodiment of a method for treating a subject described herein as well.

The delivery mechanism may be any suitable structure known in the art, such as a syringe having a needle insertable into the subject and a plunger. Instead of a syringe, other delivery mechanisms may be used for the intermittent or continuous distribution of the compositions, such as infusion pumps, syringe pumps, intravenous pumps or the like. Typically, these mechanisms include an actuating mechanism, e.g., a plunger or pump, for directing a composition into the subject. In one embodiment, a structure, e.g., catheter or syringe needle, which may be inclusive of or separate from the delivery mechanism, is first inserted into the subject and the composition is administered through the structure through activation of the actuating mechanism.

As explained herein, the compounds have been shown to exhibit exceptional anti-metastatic activity with low toxicity. Thus, in certain embodiments, the one or more anti-cancer agents of the present invention may be administered to a subject in combination with a known therapy to help block the spread of a tumor and allow time for another therapy to work on the tumor. In one embodiment, the tumor is a primary tumor. When the cancer being prevented or treated herein is pancreatic cancer, the conjunctive therapy may comprise radiation, Whipple surgery, and/or administration of chemotherapeutic agents, including targeted therapies, such as Fluorouracil, Erlotinib Hydrochloride, Gemcitabine Hydrochloride, Mitozytrex (Mitomycin C), Mutamycin (Mitomycin C), or Tarceva (Erlotinib Hydrochloride) or DFMO or combination therapies like FOLFIRINOX.

When the cancer being prevented or treated herein is breast cancer, the conjunctive therapy may comprise radiation, surgery, and/or administration of chemotherapeutic agents, including targeted therapies, such as Abitrexate (Methotrexate), Abraxane (Paclitaxel Albumin-stabilized Nanoparticle Formulation), Adriamycin PFS (Doxorubicin Hydrochloride), Adriamycin RDF (Doxorubicin Hydrochloride), Adrucil (Fluorouracil), Anastrozole, Arimidex (Anastrozole), Aromasin (Exemestane), Capecitabine, Clafen (Cyclophosphamide), Cyclophosphamide, Cytoxan (Cyclophosphamide), Docetaxel, Doxorubicin Hydrochloride, Efudex (Fluorouracil), Ellence (Epirubicin Hydrochloride), Epirubicin Hydrochloride, Exemestane, Fareston (Toremifene), Faslodex (Fulvestrant), Femara (Letrozole), Fluorouracil, Folex (Methotrexate), Folex PFS (Methotrexate), Fulvestrant, Gemzar (Gemcitabine Hydrochloride), Ixabepilone, Ixempra (Ixabepilone), Lapatinib Ditosylate, Letrozole, Methotrexate, Methotrexate LPF (Methotrexate), Mexate (Methotrexate), Mexate-AQ (Methotrexate), Neosar (Cyclophosphamide), Nolvadex (Tamoxifen Citrate), Novaldex (Tamoxifen Citrate), Paclitaxel, Paclitaxel Albumin-stabilized Nanoparticle Formulation, Tamoxifen Citrate, Taxol (Paclitaxel), Taxotere (Docetaxel), Toremifene, Tykerb (Lapatinib Ditosylate), or Xeloda (Capecitabine) or DFMO.

In one embodiment, a composition comprising the anti-tumor agents may be delivered to the subject along with another chemotherapeutic agent or therapy as is known in the art for treating the particular type of cancer. In one embodiment, the one or more anti-cancer agents described herein can be used in conjunction with other known therapeutic/cytotoxic agents. PCT application no. PCT/US10/35800 is referred to as a resource of such chemotherapeutic agents and is incorporated herein by reference. In one embodiment, the conjunctive agent comprises one or more cytotoxic chemotherapeutic agents shown to have been mutagenic, carcinogenic and/or teratogenic, either in treatment doses in in vivo or in vitro studies.

The mode of administration for a conjunctive formulation in accordance with the present invention is not particularly limited, provided that the composition comprising one or more of the anti-metastatic agents described herein and the conjunctive agent are combined upon administration. Such an administration mode may, for example, be (1) an administration of a single formulation obtained by formulating one or more of the anti-metastatic agents and the conjunctive agent simultaneously; (2) a simultaneous administration via an identical route of the two agents obtained by formulating one or more of the anti-cancer agents and a conjunctive agent separately; (3) a sequential and intermittent administration via an identical route of the two agents obtained by formulating one or more the anti-cancer agents and a conjunctive agent separately; (4) a simultaneous administration via different routes of two formulations obtained by formulating one or more of the anti-cancer agents and a conjunctive agent separately; and/or (5) a sequential and intermittent administration via different routes of two formulations obtained by formulating one or more of the anti-cancer agents and a conjunctive agent separately (for example, one or more of the anti-cancer agents followed by a conjunctive agent or its composition, or inverse order) and the like.

The dose of a conjunctive formulation may vary depending on the formulation of the one or more anti-cancer agents and/or the conjunctive agent, the subject's age, body weight, condition, and the dosage form as well as administration mode and duration. One skilled in the art would readily appreciate that the dose may vary depending on various factors as described above, and a less amount may sometimes be sufficient and an excessive amount should sometimes be required.

The conjunctive agent may be employed in any amount within the range causing no problematic side effects. The daily dose of a conjunctive agent is not limited particularly and may vary depending on the severity of the disease, the subject's age, sex, body weight and susceptibility as well as time and interval of the administration and the characteristics, preparation, type and active ingredient of the pharmaceutical formulation. An exemplary daily oral dose per kg body weight in a subject, e.g., a mammal, is about 0.001 to 2000 mg, preferably about 0.01 to 500 mg, more preferably about 0.1 to about 100 mg as medicaments, which is given usually in 1 to 4 portions.

When one or more of the anti-cancer agents and a conjunctive agent are administered to a subject, the agents may be administered at the same time, but it is also possible that the conjunctive agent is first administered and then the one or more anti-cancer agents is administered, or that the one or more anti-cancer agents is first administered and then the conjunctive agent is administered. When such an intermittent administration is employed, the time interval may vary depending on the active ingredient administered, the dosage form and the administration mode, and for example, when the conjunctive agent is first administered, the one or more anti-cancer agents may be administered within 1 minute to 3 days, preferably 10 minutes to 1 day, more preferably 15 minutes to 1 hour after the administration of the conjunctive agent. When the one or more anti-cancer agents is first administered, for example, then the one or more anti-cancer agents may be administered within 1 minute to 1 day, preferably 10 minutes to 6 hours, more preferably 15 minutes to 1 hour after the administration of the one or more anti-cancer agents.

It is understood that when referring to the one or more anti-cancer agents and a conjunctive agent, it is meant the one or more anti-cancer agents alone, a conjunctive agent alone, as a part of a composition, e.g., composition, which optionally includes one or more pharmaceutical carriers. It is also contemplated that more than one conjunctive agent may be administered to the subject if desired.

Cancer cells rely upon nutrients to fuel their rapid growth and survival in vivo. Compounds which deplete amino acid pools can, therefore, starve these tumors of the biomolecules needed to sustain them and as a result inhibit their growth. Various embodiments describe herein relate to novel compounds which decrease intracellular leucine and methionine levels. For example, as a result of treatment with these inhibitors, the intracellular levels of methionine decrease which in turn affects many metabolic processes which rely upon methionine and its derivatives. For example, depleted methionine levels limit S-adenosylmethionine formation and, in turn, halt polyamine biosynthesis and significantly reduce intracellular pools of the native polyamines, spermidine and spermine. Various embodiments provide novel compositions of matter which reduce intracellular amino acid levels and provide a new way to treat human cancers via nutrient deprivation.

Without wishing to be bound by theory, it is believed that the compounds according to various embodiments are targeting LAT-1, an amino acid transporter used to import leucine, phenylalanine and methionine. There are several existing LAT-1 amino acid uptake inhibitors and only one is in clinical trials to date. All known LAT-1 inhibitors have alpha amino acid (functional groups) within their structures and are mostly phenylalanine derivatives. The structural designs of various embodiments are unique compositions of matter and are very different and are potentially more potent than current LAT-1 inhibitors in terms of depleting cells of methionine. Unlike the other LAT-1 inhibitors these compounds work by inhibiting import and stimulating amino acid efflux from cells. Also, the compounds of various embodiments contain hydrophobic residues, which may further facilitate their uptake. With that said, there may be other mechanisms by which the amino acids are depleted in the cells.

The materials, according to various embodiments, may have applications in treatment of human diseases as anticancer agents or as anti-infectives, especially for tropical diseases involving parasitic infections as these microorganisms may be very sensitive to nutrient deprivation approaches (e.g., malaria, tuberculosis).

The approach, according to various embodiments, presents the native amino acid side chains (or side chains that closely resemble the native side chains of amino acids) in a quasi-symmetrical array, where the side chains of leucine and phenylalanine are presented on both ends of the inhibitor molecule. These molecular side chains when presented in this fashion markedly accelerate the depletion of methionine resources to the point where intracellular methionine levels become virtually undetectable. As a result, this approach only requires low micromolar levels of Compound 10 (1666.255) to affect cell growth of pancreatic cancer cells.

Various embodiments provide a potential anticancer drug at the outset due to its potent anti-growth effect on a very aggressive pancreatic ductal adenocarcinoma (PDAC) cell line (i.e., L3.6pl cells). Pancreatic cancer is expected to become the second leading cause of cancer related death by 2030, and existing medicines only modestly extend life for 6-11 months. Thus, the compounds and techniques according to various embodiments meet a desperate clinical need.

Methionine depletion should also affect other cell metabolites including the native polyamines: spermidine (Spd), and spermine (Spm). These polyamines, along with putrescine (Put), are important growth factors in eukaryotic cells.¹ At physiological pH, the native polyamines are fully protonated, allowing them to interact with anions in the cell including nucleic acids, proteins, and phospholipids. Polyamines are involved in many biological processes, such as cell replication, translation, transcription, and regulation of specific gene expression.¹⁻² In addition, they have roles in the regulation of cell proliferation, apoptosis, and tumorigenesis. An association between high levels of polyamines and rapid proliferation of eukaryotic cells and cancer was reported in 1968 by Russell and Snyder.³ Tumor cells in particular accumulate high polyamine concentrations, particularly spermidine, and typically exhibit a high ratio of spermidine to spermine.³⁻⁴ Depletion of intracellular spermidine and spermine has been shown to cause an arrest in cell growth through the inhibition of translation.⁵ Polyamine depletion also inhibits DNA synthesis and affects the number of growth-regulating genes, which results in growth arrest. Thus, maintenance of polyamine homeostasis is critical for cell viability and proliferation.⁶ The ability to modulate polyamine pools via methionine depletion using the embodiments described herein provides a powerful method to control cell growth.

Spermidine and spermine biosynthesis requires the addition of an aminopropyl group onto a putrescine or spermidine substrate, respectively.¹ This aminopropyl group is derived from L-methionine. Specifically, L-methionine is converted to S-adenosyl-L-methionine (SAM) via methionine adenosyltransferase (MAT). SAM is then decarboxylated by S-adenosylmethionine decarboxylase (AdoMetDC) to form S-adenosylmethioninamine, i.e., decarboxylated AdoMet.⁹ Two aminopropyltransferases, spermidine synthase (SRM) and spermine synthase (SMS), transfer an aminopropyl moiety from S-adenosylmethioninamine to their respective substrates (putrescine or spermidine) to form spermidine or spermine. Therefore, rapidly dividing cells must convert some their intracellular methionine pools towards SAM and S-adenosylmethioninamine (decarboxylated AdoMet) formation to drive polyamine biosynthesis. In summary, polyamine biosynthesis is directly linked to amino acid (L-ornithine and L-methionine) availability.

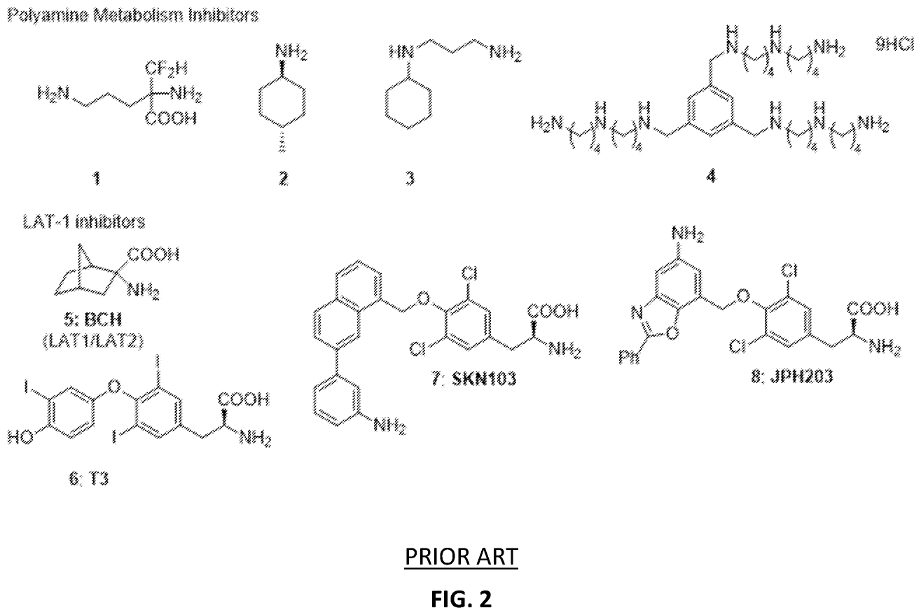

Polyamine homeostasis is maintained through a balance of polyamine biosynthesis, degradation, uptake and excretion.⁷ The first step in polyamine biosynthesis is the formation of putrescine from ornithine by ornithine decarboxylase (ODC). The amino acid L-ornithine itself can be generated from L-arginine (via arginase) or be imported from the plasma. Due to its short half-life (10-30 minutes in mammalian systems), ODC is regulated at multiple steps from transcription to post-translational modification.¹ ODC activity is often upregulated in human cancers relative to surrounding normal tissues⁸ in an effort to increase intratumoral polyamine pools through the biosynthetic pathway. As such, ODC is a well-established cancer target. Indeed, α-difluoromethylornithine (DFMO) was developed as an irreversible inhibitor of ODC that suppresses cancer development in animal models.⁷ Treatment with DFMO typically results in rapid depletion of intracellular putrescine and spermidine, and growth arrest. 5 Cancers often circumvent DFMO by upregulating polyamine transport to replenish their polyamine pools. Polyamine transport inhibitors (PTIs) have been developed to address this DFMO escape pathway.⁷ For example, L3.6pl human pancreatic cancer cells treated with DFMO+PTI (see example PTI structure 4 in FIG. 2) in the presence of exogenous spermidine (Spd, 1 μM) remained polyamine depleted and had decreased viability, whereas those treated with DFMO+Spd were >90% viable and had increased intracellular polyamine pools.⁷

Polyamine catabolism involves spermine/spermidine N¹-acetyltransferase (SAT1), which catalyzes the formation of N¹-acetylspermine and N¹-acetylspermidine by transferring the acetyl moiety from acetyl-coenzyme A (acetyl-CoA) to the N¹ position of spermine or spermidine. Acetylpolyamine oxidase (APAO) then catalyzes the conversion of these acetylated polyamines to spermidine or putrescine, respectively, via oxidative cleavage.² Note: spermine oxidase (SMOX) can directly convert spermine directly to spermidine.¹ In addition to being converted to spermidine or putrescine, the N-acetylated polyamine products of SAT1 reactions are also exported from the cells. In this regard, cells have the ability to maintain polyamine homeostasis though modulation of polyamine biosynthesis, transport, and catabolization.⁸

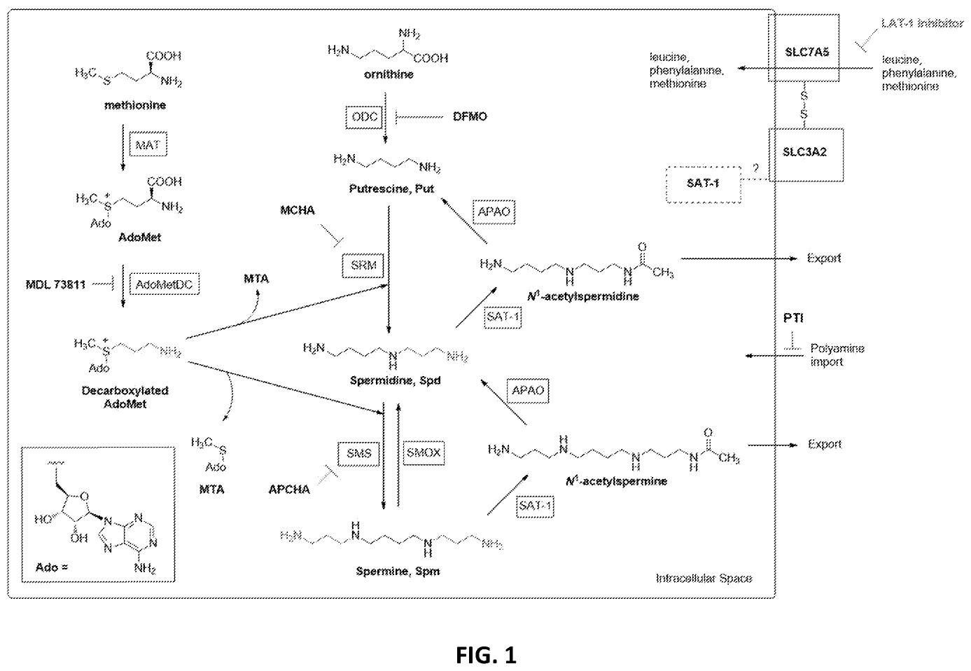

FIG. 1 is an example according to various embodiments, illustrating polyamine metabolism and methionine supply. Putrescine is formed by ornithine decarboxylase (ODC) as the first step in polyamine biosynthesis. ODC can be inhibited by the suicide inhibitor α-difluoromethylornithine (DFMO). Methionine is converted to S-adenosylmethionine (AdoMet) by methionine adenosyltransferase (MAT). S-adenosylmethionine decarboxylase (AdoMetDC) provides decarboxylated AdoMet for construction of the higher polyamines via aminopropylation. Note: AdoMetDC is inhibited by MDL 73811. Decarboxylated AdoMet provides the aminopropyl donor for the synthesis of spermidine and spermine via spermidine synthase (SRM) and spermine synthase (SMS), respectively. Trans-4-methylcyclohexylamine (MCHA) and N-(3-aminopropyl)-cyclohexylamine (APCHA) inhibit spermidine and spermine synthase, respectively. Spermine oxidase (SMOX) converts spermine back to spermidine directly. In contrast, spermine/spermidine N¹-acetyltransferase (SAT-1) catalyzes the formation of N-acetylspermine and N-acetylspermidine. These acetylated polyamines can then be exported from the cell or converted to the lower polyamines by acetylpolyamine oxidase (APAO). Polyamines can be imported into cells via the polyamine transport system, which can be blocked through the use of a polyamine transport inhibitor (PTI). SLC7A5 (solute carrier 7A5, LAT-1) and SLC3A2 (solute carrier 3A2) form a heterodimer known as LAT-1/SLC3A2 (large neutral amino acid transporter 1) and transport neutral amino acids (e.g., leucine, phenylalanine and methionine) into cells.

There is a great need to develop inhibitors of polyamine metabolism. In addition to DFMO (1), trans-4-methylcyclohexylamine (MCHA, 2) and N-(3-aminopropyl)-cyclohexylamine (CDAP, 3) have been developed as potent competitive inhibitors of spermidine and spermine synthases, respectively.¹⁰ These compounds, shown in FIG. 2, effectively inhibit their specific targets. However, cancer cells can modulate/interconvert their polyamine pools or increase polyamine uptake to address these interventions.¹¹ PTIs like the one shown in FIG. 2 (compound 4) have been shown to be effective in depleting cells of polyamines when used in combination with DFMO even in the presence of exogenous spermidine.⁷ Indeed, this combination of a polyamine biosynthesis inhibitor and a PTI has been shown to significantly increase survival in an orthotopic mouse model of pancreatic cancer using murine PanO2 cells.¹²

Beyond inhibiting the polyamine biosynthetic enzymes, another approach is to target the amino acid pools from which the polyamines are derived. LAT-1/SLC3A2 (large neutral amino acid transporter 1) is a heterodimer comprised of a light subunit (SLC7A5, aka LAT1) and a heavy subunit (SLC3A2). This complex transports large neutral amino acids such as leucine and phenylalanine as well as methionine. L-Leucine is used not only for protein synthesis, but also serves as an intracellular signaling molecule, which can regulate cell growth via stimulation of the mechanistic/mammalian target of rapamycin (mTOR). Once activated, mTOR directly phosphorylates initiation factor 4E binding protein (4E-BP1) and p70 ribosomal S6 kinase 1 (p70S6K) to facilitate growth.¹³ Activation of the mTOR pathway is found in many types of cancers and inhibitors of LAT-1 (5-8) and mTOR have been proposed as an anticancer strategy.¹³ Indeed, inhibition of LAT-1 has been shown to suppress leucine uptake, mTOR signaling and the growth of Panc-1 pancreatic cancer cells in vitro.¹³ Compounds which cause intracellular methionine (and/or leucine) depletion via efflux mechanisms are also expected to affect cell growth.

FIG. 2 is an example according to various embodiments, illustrating prior art inhibitors of polyamine metabolism (1-3), polyamine import (4) and LAT-1 (5-8). Note: existing LAT1 inhibitors (5-8) are all predicated upon alpha amino acid designs.

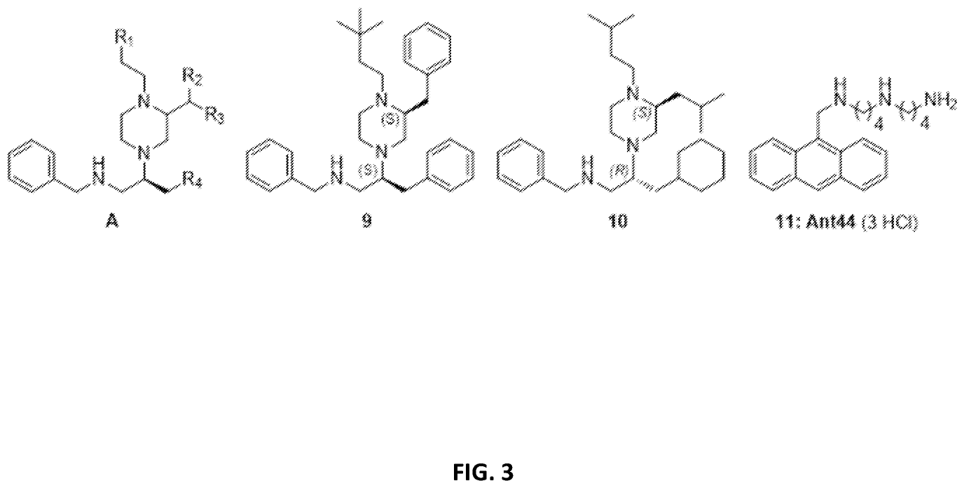

In a search for compounds which decrease intracellular polyamine levels, development of various embodiments involved screening several molecular libraries from the Torrey Pines Institute for Molecular Studies (TPIMS) and identified a lead architecture A for further investigation (see A in FIG. 3). In a subsequent study of 250 individual compounds, two promising hits (compounds 9 and 10 in FIG. 3) were identified. Various embodiments relate to the synthesis and bioevaluation of these hit compounds, which appear to deplete cells of methionine. L3.6pl pancreatic cells treated in vitro with compound 10 (5 μM) were shown to have significant levels of glutamic acid, agmatine (a derivative of arginine), and ornithine in the supernatant and have significantly decreased intracellular leucine, methionine, spermidine and spermine pools. Various embodiments provide new ways to deplete polyamine pools and influence cell growth via decreased intracellular methionine.

FIG. 3 is an example according to various embodiments, illustrating lead architecture (A) identified from molecular library screening, top hits 9 and 10, and 11 (Ant44, a fluorescent cytotoxic polyamine).

Since the original TPIMS molecular libraries were synthesized via solid-phase peptide chemistry¹⁴ to provide small quantities for screening purposes, various embodiments relate to the development of a solution phase synthetic approach to provide larger quantities for further evaluation of the top hits.

As a quick overview, a strategy according to various embodiments (shown in Scheme 1) for synthesizing compound 9 involved several peptide coupling steps with HATU (1-[Bis(dimethylamino)methylene]-1H-1,2,3-triazolo[4,5-b]pyridinium-3-oxide hexafluoro-phosphate) to create a linear triamide scaffold 17 with the appropriate substituents. This triamide scaffold was then reduced with borane-THF to afford the respective chiral triamine 18. Regioselective N-benzoylation of the terminal amine with N-hydroxysuccinimide ester 19 provided a diamine scaffold 20, which could then be regiospecifically cyclized with oxalyl diimidazole to form diketopiperazine 21. Lastly, 21 could be reduced with BH₃/THF to form the desired target compound 9.

As an example, the synthesis of compound 9 (1666.177) is shown in Scheme 1. The starting materials 3,3-dimethyl-butyric acid 12 and L-phenylalanine methyl ester hydrochloride 13 were used in the initial coupling step to form amide 14 (177-1, 95%), which was then hydrolyzed to form carboxylic acid 15 (99% yield). HATU-mediated coupling of acid 15 (177-2) to L-phenylalanine amide 16 gave triamide 17 (177-3) albeit in low yield (39%). Reduction of the triamide using BH₃/THF provided the triamine 18 in 52% yield. Subsequent N-benzoylation with N-hydroxysuccinimide ester of benzoic acid 19 gave the desired benzamide 20 in 90% yield. Cyclization of 20 with oxalyl diimidazole provided the diketopiperazine 21 (79% yield), which was reduced with BH₃/THF to give the desired compound 9 (1666.177, 65% yield). Overall, compound 9 was made in 9% yield over 7 steps.

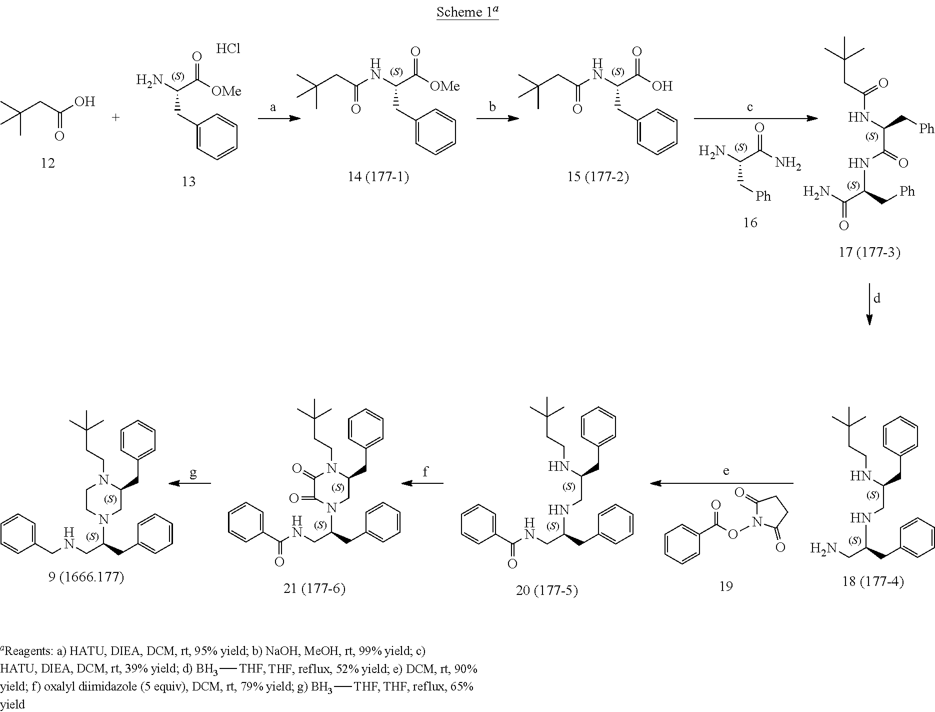

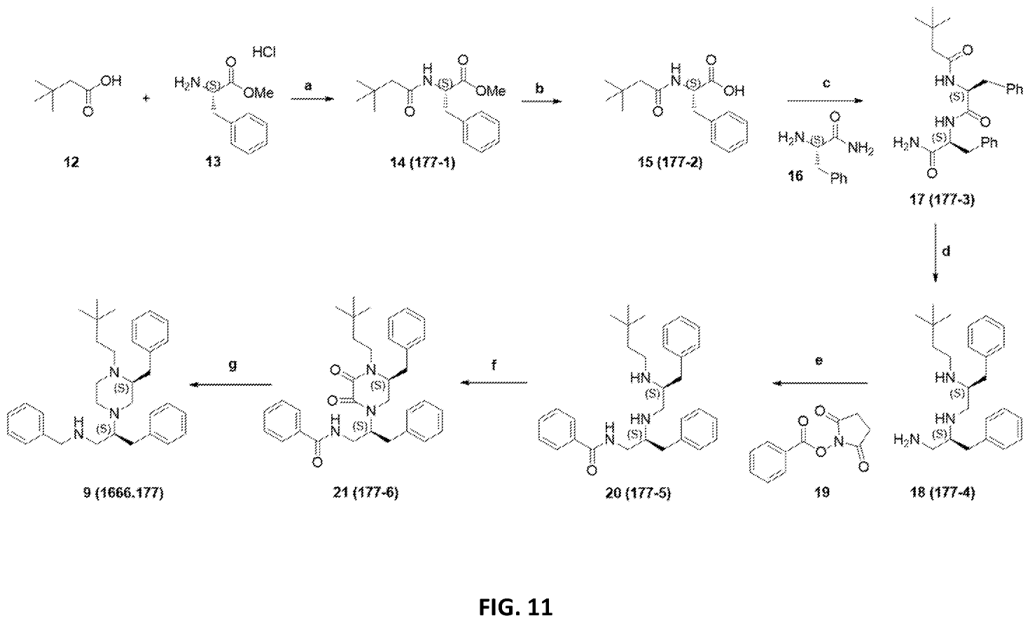

FIG. 11 is an example according to various embodiments, illustrating an enlargement of Scheme 1.

Compound 10 was synthesized using a modified approach to increase yield. As shown in Scheme 2, isovaleric acid 22 and L-leucine ether ester hydrochloride 23 were coupled via HATU to form amide 24 as the initial step in the synthesis of target 10 (1666.255). Amidoester 24 was then converted to the carboxylic acid 25 in high yield. To avoid the low yields encountered in the prior coupling of acid 15 with amide 16, various embodiments involved first coupling acid 25 to D-cyclohexylalanine methyl ester hydrochloride 26 to give ester 27 (255-3) in 96% yield. Ammonia gas was used to convert the diamidoester 27 (255-3) to triamide 28 (255-4) in 82% yield. Note: this alternate approach to triamide formation (e.g., 28) was a significant improvement over the approach used to make triamide 17. The remainder of the synthesis was carried out as described above to give the target compound 10 (1666.255) in 22% overall yield after 8 steps.

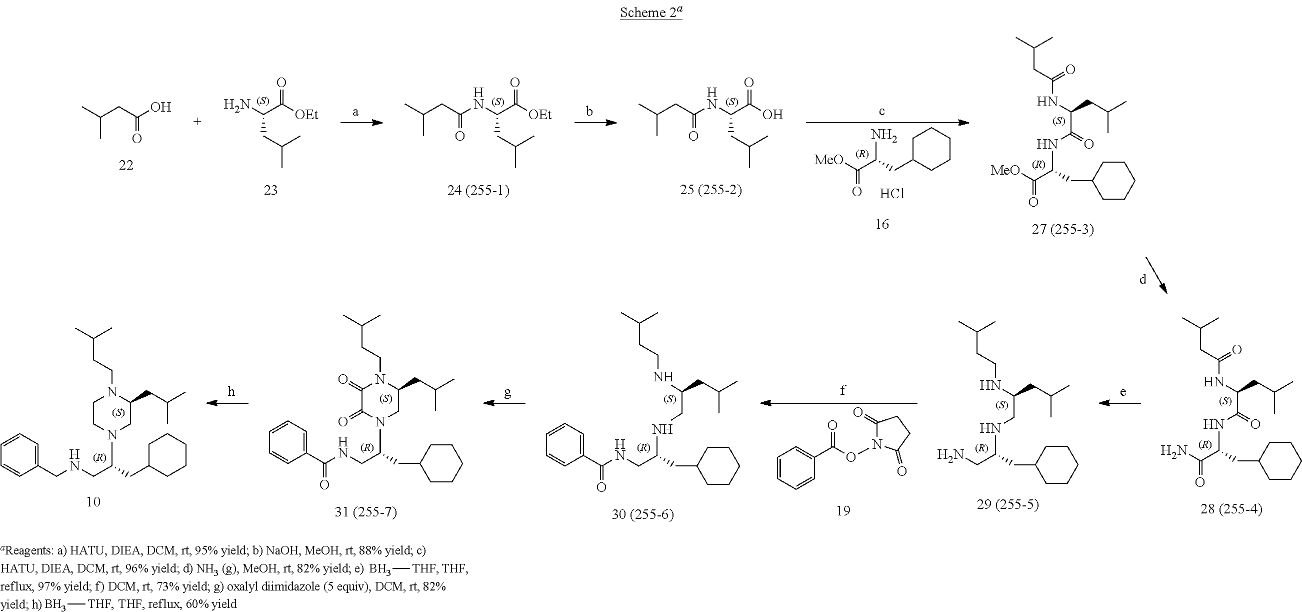

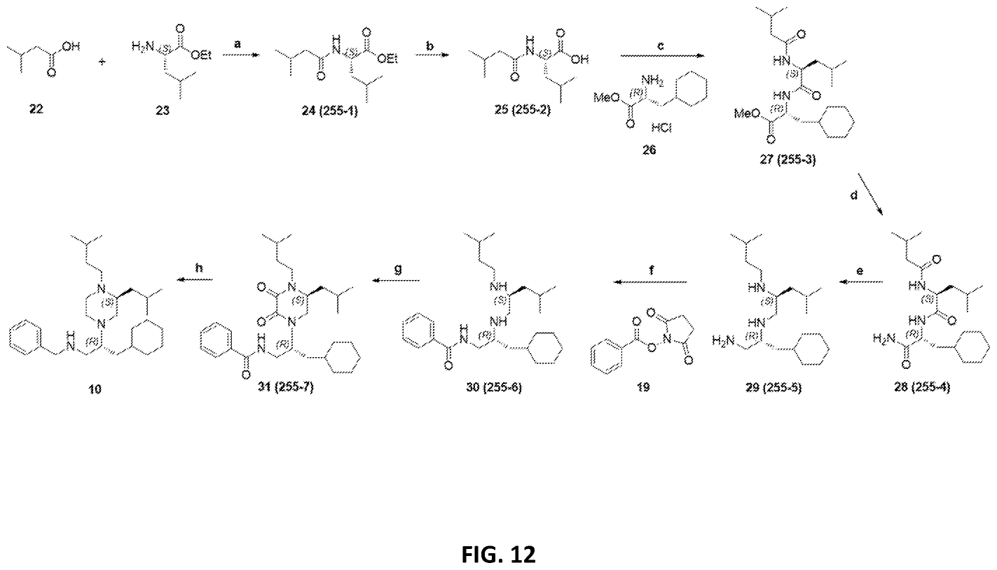

FIG. 12 is an example according to various embodiments, illustrating an enlargement of Scheme 2.

The bioevaluation of these compounds was then conducted using a series of assays designed to compare the ability of each compound to target polyamine metabolism and reduce intracellular levels of key amino acids and polyamines.

CHO K1 Studies. Wild type Chinese hamster ovary (CHO K1) cells were chosen to first study the synthetic compounds' impact on polyamine metabolism. The CHO K1 cell line has high polyamine transport activity and was useful in screening compounds for their polyamine transport inhibitor activity. A dose-response curve was obtained for each compound to determine their toxicity in CHO K1 cells after 48 h incubation. Compound 9 (1666.177) was non-toxic up to the highest dose tested (15 μM). In contrast, compound 10 (1666.255) had a very sharp cytotoxicity curve and a 48 h IC₅₀ of 10.8±0.22 μM. Interestingly, compound 10 could be dosed for 48 h at ≤10 μM in CHO-K1 cells without apparent toxicity suggesting that a critical concentration of 10 was needed to affect growth.

Inhibition of ODC by DFMO often leads to an increase in polyamine transport activity to maintain intracellular polyamine homeostasis (see FIG. 1). The increased transport activity of DFMO-treated cells was used to assess the polyamine transport inhibitor (PTI) activity of these compounds by investigating the ability of each compound to block the entry of a rescuing dose of spermidine (1 μM).¹⁵⁻¹⁶ Our group has previously determined the 48 h IC₅₀ value of DFMO in CHO K1 cells as 4.2 mM, as well as the minimum amount of spermidine (Spd, 1 μM) required to rescue the DFMO-treated CHO K1 cells back to >90% viability.⁷ These two parameters (4.2 mM DFMO and 1 μM Spd) remained fixed throughout the assay. The third parameter was the candidate compound at increasing doses up to its maximum tolerated dose, MTD, which was the maximum dose the compound could be dosed individually and provide % viability >90% relative to an untreated control. Since non-toxic PTI compounds are expected to inhibit Spd entry, the cells treated with a combination of DFMO, Spd, and PTI would be expected to resemble the DFMO-only treated control. This assay allowed the potential PTIs to be tested, ranked and compared.

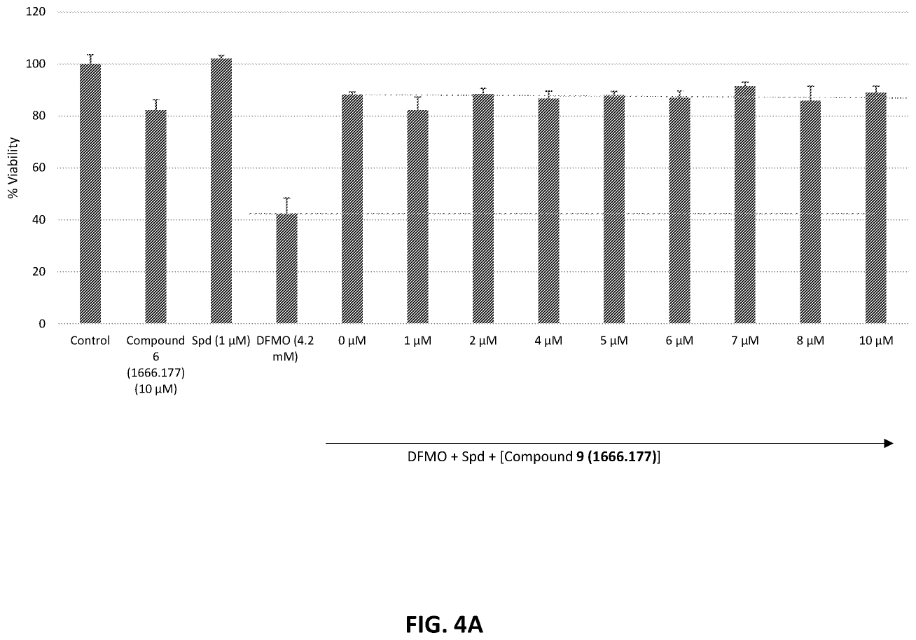

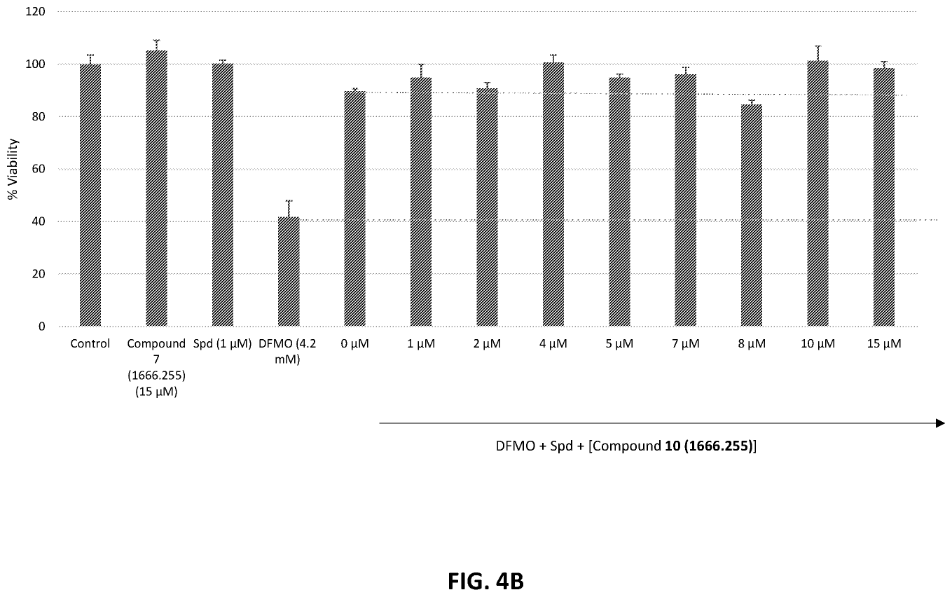

In a 96-well plate, CHO K1 cells were treated with the IC₅₀ of DFMO (4.2 mM), a fixed dose of Spd (1 μM), and increasing doses of the potential PTI compounds (0 to 10 μM). The cells were incubated for 48 h at 37° C. Results for compounds 9 (1666.177), and 10 (1666.255) are shown in FIG. 4. The green line in FIG. 4 represents the % viability observed with the DFMO+Spd control, while the red line represents the % viability for the DFMO-only control. The EC₅₀ value is defined here as the concentration of the compound needed to reduce the % viability to halfway between the green and red lines, i.e. halfway between the DFMO+Spd and DFMO only controls. Interestingly, neither of the compounds (9 or 10) successfully blocked the entry of the rescuing dose of Spd to the DFMO-treated CHO K1 cells and the EC₅₀ could not be determined. In summary, these CHO K1 experiments demonstrated that compounds 9 and 10 did not act as PTIs in this cell line.

FIG. 4A is an example according to various embodiments, illustrating the inability of Compound 9 (1666.177) to prevent Spd from rescuing DFMO-treated CHO K1 cells. FIG. 4B is an example according to various embodiments, illustrating the inability of Compound 10 (1666.255) to prevent Spd from rescuing DFMO-treated CHO K1 cells. The cells were incubated at 37° C. for 48 h in the presence of increasing doses of the respective compound in the presence of a fixed concentration of DFMO (4.2 mM) and Spd (1 μM). The cells were incubated with 1 mM aminoguadine (AG) for 24 h prior to compound addition. Column 1 shows the untreated CHO K1 control, while column 2 shows the % cell viability when the cells are dosed with the compound alone at the highest concentration tested and shows the compounds as nontoxic. Columns 3 and 4 shows the Spd only control at 1 μM and DFMO only control at 4.2 mM respectively. Columns 5-13 are fixed concentrations of DFMO (4.2 mM) and Spd (1 μM) with increasing concentrations of the compounds indicated in each panel. The data suggests that neither compound performs as a PTI and are affecting cell growth through another mechanism (e.g., methionine depletion).

Nevertheless, to further assess PTI activity, development of various embodiments also involved screening the candidate compounds (9 and 10) against Ant-44 entry (compound 11). Ant-44 is a cytotoxic homospermidine-anthracene conjugate previously synthesized. Ant-44 is taken up into CHO K1 cells through the polyamine transport system (PTS). The selectivity for the PTS was demonstrated through IC₅₀ comparisons between the CHO cell line and a mutant CHO cell line (CHO-MG). The CHO-MG cell line is a polyamine-transport-deficient cell line and represented cells with low PTS activity. Ant-44 displayed a nearly 150-fold preference for the CHO cell line over the CHO-MG, suggesting that Ant-44 has high affinity for targeting cells with active polyamine transport activity.¹⁷ Additionally, the presence of spermidine provides cell protection from the polyamine conjugate Ant-44 via spermidine's competitive access to cells via the PTS.¹⁸ Based on this result, it was concluded that a PTI, especially a polyamine-based PTI like compound 4 in FIG. 2, would also block Ant-44 uptake.

Various embodiments involve the concept that a non-toxic PTI agent would inhibit the uptake of the cytotoxic polyamine conjugate Ant-44 (11) and rescue cells from Ant-44 induced toxicity. For example, using the IC₅₀ dose of Ant-44, PTIs could be identified by measuring a compound's ability to block Ant-44 entry and rescue cells back to >90% viability. Previous studies demonstrated that Ant-44 (2.4 μM) significantly reduced cell viability in CHO K1 cells after 48 h incubation. This toxic dose of Ant-44 was kept constant throughout the assay, while the candidate PTI compound was added in increasing concentrations up to its MTD.

Compounds (9 and 10) were tested at 5 μM and 7 μM. CHO K1 cells were treated in a 96-well plate with a toxic dose of Ant-44 (2.4 μM) alone and dosed with the candidate compounds (at 5 μM and 7 μM) and incubated for 48 h at 37° C. Compounds 9 (1666.177) and 10 (1666.255), exhibited intriguing activity. Rather than rescue the cells from Ant-44, the compounds seemed to significantly potentiate Ant-44's toxicity. For example, Ant-44 alone (2.4 μM) gave 22.5% viability, whereas Ant-44 in combination with compounds 9 (1666.177) or 10 (1666.255) at 7 μM gave significantly reduced relative viability at 2.1% and 3%, respectively, compared to the untreated control. Since neither 9 or 10 was toxic below 10 μM in CHO K1 cells, this result implied synergism between these compounds and Ant-44.

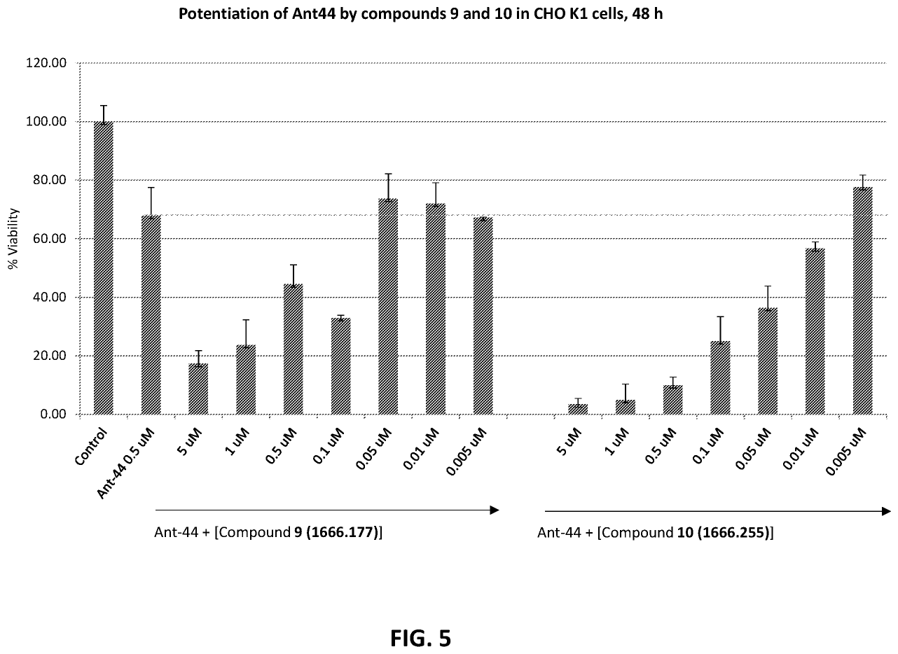

To further explore this effect, development of various embodiments included modifying an original screen to use a lower dose of Ant-44 (0.5 μM) to improve the dose range of 9 and 10 that could be tested. In this regard, Ant-44 was dosed at 0.5 μM alone and in combination with increasing doses of compounds 9 (1666.177) and 10 (1666.255) and the CHO K1 cells were incubated for 48 h at 37° C., and the results are shown in FIG. 5. The red line represents the % cell viability of the Ant-44 only control. The Ant44 potentiation assay EC₅₀ value is defined as the concentration of the candidate compound required to decrease the cell viability to half that of the Ant-44 only control. Both compounds 9 and 10 were effective at decreasing cell viability, when used in combination with Ant-44 in a dose dependent fashion. Additionally, they exhibited very low EC₅₀ concentrations in CHO cells in the presence of Ant-44 (0.5 μM), with EC₅₀ values of 750 nM and 60 nM, respectively. Compound 10 (1666.255) was approximately 12.5 times more effective at potentiating Ant-44 than compound 9 (1666.177) in CHO K1 cells.

FIG. 5 is an example according to various embodiments, illustrating potentiation of Ant-44 toxicity by compounds 9 and 10 in CHO K1 cells. Cells were incubated for 48 h at 37° C. with the respective compound and a fixed concentration of cytotoxic Ant-44 (0.5 μM). A 1 mM AG solution was incubated with the CHO K1 cells for 24 h prior to the addition of candidate compound. This was necessary to protect Ant-44 from the amine oxidases present in the media containing fetal bovine serum. Column 1 is the untreated CHO K1 control cells, column 2 shows the % cell viability when dosed with Ant-44 alone at 0.5 μM, columns 3-16 have a fixed concentration of Ant-44 (0.5 μM) with decreasing concentrations of the candidate compounds as indicated in each lane. Both compounds are nontoxic at the highest concentration tested (5 μM). The Ant-44 potentiation EC₅₀ values, defined as the concentration to reduce the viability to half the Ant-44 only control, were 0.75 μM (9) and 0.06 μM (10), respectively.

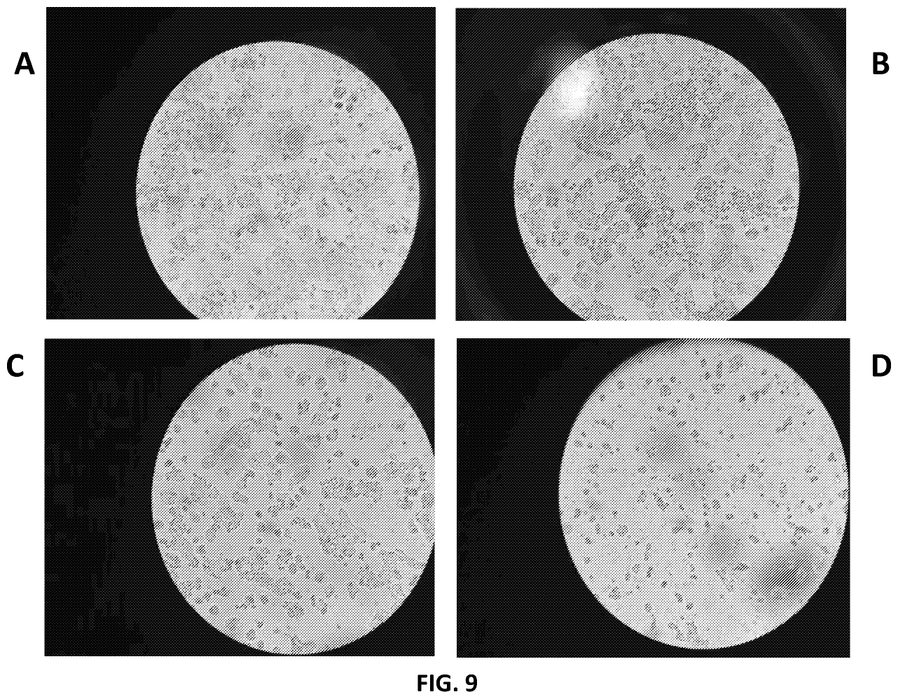

To observe changes to the cell as a result of treatment with these compounds, the control CHO K1 cells and the cells treated with Ant-44 (0.5 μM) and compound 10 (1666.255) at 5 μM in the aforementioned 96-well plate experiment were observed under the microscope. As shown in FIG. 9, the treated cells were not ruptured, and differed from the control in terms of their number and rounded shape (in comparison to the elongated control cells). This data suggested that the treated cells were not growing. Next, development of various embodiments involved looking at the L3.6pl human pancreatic cancer cell line.

L3.6pl Studies. Compounds 9 and 10 were evaluated in the metastatic human pancreatic cancer cell line, L3.6pl. L3.6pl has a K-ras mutation, and high polyamine uptake activity.⁷ A dose-response curve was obtained for each compound in L3.6pl cells to determine the 72 h L3.6pl IC₅₀ value, described as the dose at which L3.6pl cells were 50% viable compared to the untreated control. Compounds 9 (1666.177) and 10 (1666.255) had 72 h IC₅₀ values of 11.7±0.9 μM and 5.9±0.2 μM respectively. The fact that compounds 9 and 10 were more toxic to L3.6pl cells compared to CHO K1 cells suggested that there may be enhanced targeting of cancer cell types.

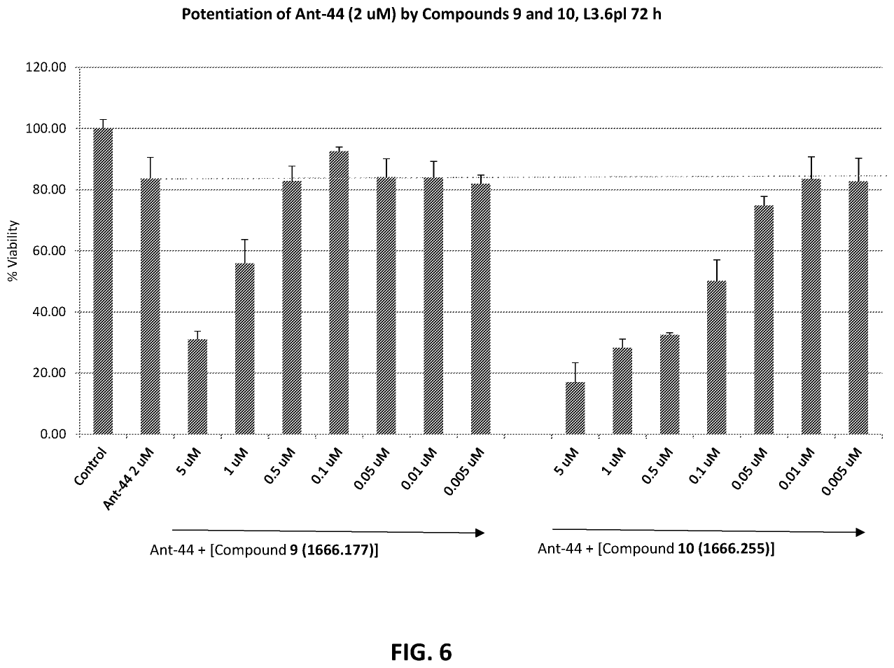

As performed previously with CHO K1 cells, L3.6pl cells were treated with compounds 9 (1666.177) and 10 (1666.255) and a fixed dose of Ant-44 to observe the potentiation effect. The 72 h IC₅₀ dose of Ant-44 in L3.6pl cells was previously determined to be 4 μM. For this study, half that dose was used to replicate the large window used in the CHO experiments to look at reduction in cell viability. In a 96-well plate, L3.6pl cells were dosed with a fixed concentration of Ant-44 (2 μM) and increasing doses of compounds 9 (1666.177) and 10 (1666.255). The cells were incubated for 72 h at 37° C., and the results are given in FIG. 6. Although both compounds were effective at reducing cell viability, higher doses were required compared to CHO K1 cells. The EC₅₀ for compound 9 (1666.177) was 3.27±0.17 μM and compound 10 (1666.255) was 0.29±0.1 μM. Both compounds were effective well below their 72 h IC₅₀ dose in L3.6pl cells. Similar to the observations in CHO K1 cells, compound 10 (1666.255) was approximately eleven times more effective at increasing the potency of Ant-44 in L3.6pl cells than compound 9 (1666.177).

FIG. 6 is an example according to various embodiments, illustrating the ability of compounds 9 (1666.177) and 10 (1666.177) to potentiate the effect of Ant-44 in L3.6pl cells. Cells were incubated for 72 h at 37° C. with the respective compound and Ant-44 (2 μM). A 250 μM AG solution was incubated with the cells for 24 h prior to addition of compounds. Column 1 is the untreated L3.6pl control cells, column 2 shows the % cell viability when dosed with Ant-44 alone at 2 μM, columns 3-16 have a fixed concentration of Ant-44 (2 μM) with increasing concentrations of the candidate compounds as indicated in each lane. Both compounds are nontoxic at the second highest concentration tested (1 μM).

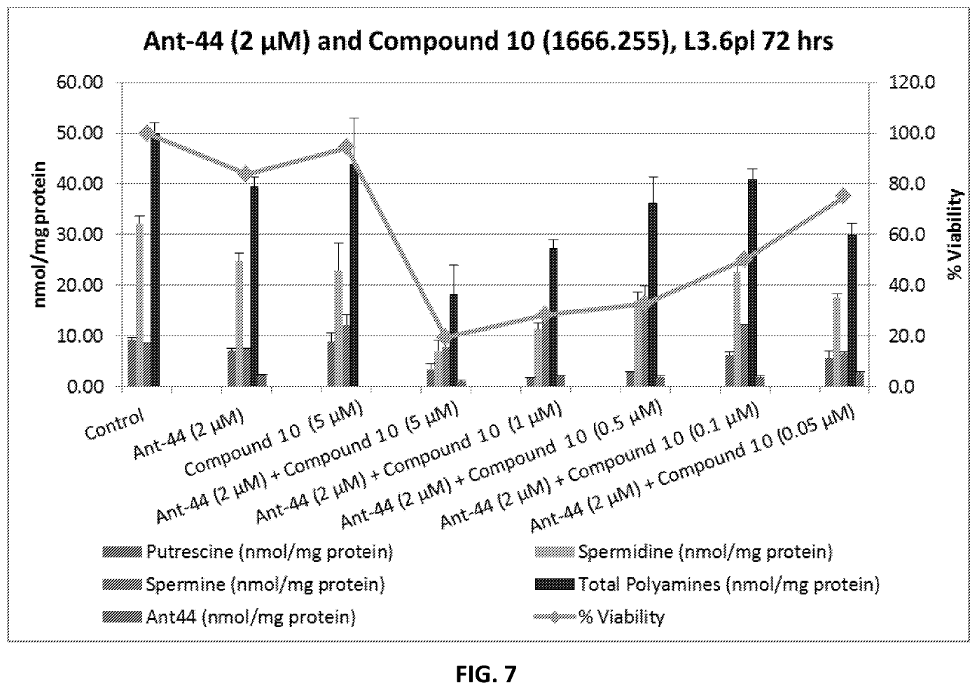

To understand why Ant-44 becomes more potent in the presence of these compounds, especially in the presence of compound 10 (1666.255), development of various embodiments involved designing an experiment to relate toxicity to intracellular polyamine and Ant-44 levels. One explanation for the enhanced potency was that compound 10 (1666.255) increased polyamine import and, as a result, may have increased intracellular Ant-44 levels. To test this hypothesis, L3.6pl cells were dosed with a fixed concentration of Ant-44 (2 μM) alone and in combination with increasing concentrations of compound 10 (1666.255) to explore how this combination therapy affected intracellular polyamine pools and Ant-44 import. These results are displayed in FIG. 7.

FIG. 7 is an example according to various embodiments, illustrating both single and combination therapies in L3.6pl cells with Ant-44 and compound 10 (1666.255) after 72 h incubation. Polyamine and Ant-44 levels (expressed as nmoles/mg protein) and relative % viability versus an untreated control were observed after 72 h incubation at 37° C. The untreated control was run in parallel and polyamine levels determined in duplicate and % cell viability in triplicate. Ant-44 was dosed at a fixed concentration of 2 μM and compound 10 (1666.255) at increasing concentrations. Cell viability tracked well with total intracellular polyamine levels (sum of putrescine, spermidine and spermine).

As shown in FIG. 7, neither Ant-44 (2 μM) or compound 10 (1666.255 at 1 μM) alone significantly reduced cell viability or intracellular polyamine pools. The intracellular level of Ant-44 was relatively unchanged, where the Ant-44 only control gave 2.15±0.10 nmol Ant44/mg protein and when compound 10 at 1 μM was present at 2.08 nmol Ant44/mg protein. If compound 10 (1666.255) was acting as a polyamine import agonist, the level of Ant-44 in the cells would be expected to increase with increasing doses of compound 10. However, this was not observed.

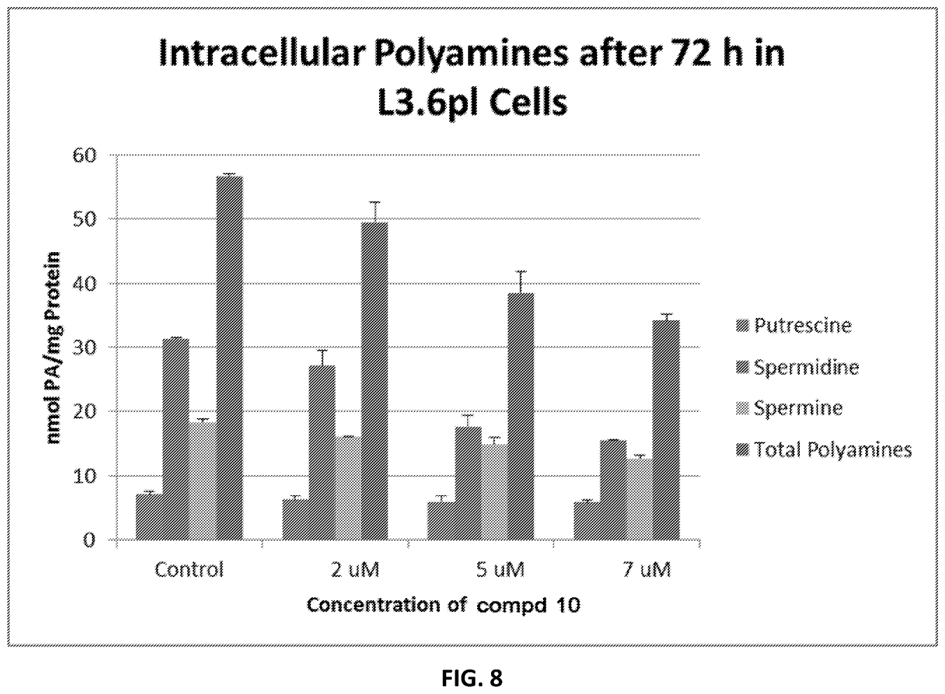

It was concluded that compound 10 was neither acting as a PTI nor as a polyamine import agonist. How then could it lead to intracellular polyamine depletion? The decreased levels of intracellular polyamines (putrescine, spermidine, and spermine) when L3.6pl cells are dosed with Ant-44 in combination with compound 10 at 1 μM suggested that compound 10 may act via a different mechanism. To test this hypothesis, L3.6pl cells were dosed with increasing concentrations of compound 10 (1666.255) without Ant44. If compound 10 (1666.255) was acting on polyamine pools, one should see a reduction in total polyamine levels as well as decreased cell viability in a dose dependent manner upon increasing levels of 10. Therefore, the toxicity of compound 10 and intracellular polyamine levels after 72 h exposure to 10 were measured (FIG. 8). The results of these experiments at 72 h are shown in FIG. 8.

FIG. 8 is an example according to various embodiments, illustrating intracellular polyamine levels (expressed as nmoles polyamine/mg protein) in L3.6pl cells dosed with increasing concentrations of compound 10 (1666.255) after cells were incubated for 72 h at 37° C. The untreated control was run in parallel and polyamine levels determined in duplicate via N-dansylation and HPLC. The data was averaged and reported as nmol polyamine (PA)/mg protein. Compound 10 demonstrated increasing toxicity to L3.6pl cells over extended periods of incubation. As shown in FIG. 8, after 72 h of incubation the intracellular polyamine levels of spermidine and spermine were significantly reduced, whereas the putrescine content was relatively unaffected.

This was interesting because typically another polyamine depletion approach (DFMO+PTI treatment) led to an absence of putrescine and a significant reduction in spermidine pools while the spermine pools were maintained. A systematic study of polyamine biosynthesis inhibitors (using DFMO, MCHA and CDAP) in L3.6pl cells revealed the plasticity of polyamine homeostasis in these pancreatic cancer cells. The cells maintained viability as long as either the spermine or spermidine pools were maintained near basal levels and as long as the total polyamine pools were >40% of the untreated control. This suggested that these cells maintain an excess pool of polyamines to help offset changes in intracellular polyamine levels. For example, L3.6pl cells were 100% viable in the presence of the SMS inhibitor (CDAP, 100 μM) and had no detectable spermine.¹⁰ Since compound 10 gave specific depletion of both spermidine and spermine pools (FIG. 8), it works through a different mechanism than DFMO+PTI.

Table 1 shows Intracellular Polyamine levels (in nmol polyamine/mg protein) after 72 hr exposure to compound 10 at increasing concentrations in L3.6pl Cells. As shown in table 1, compound 10 leads to significant dose dependent decreases in total polyamines as well as spermidine and spermine levels.

TABLE 1

| | | | | Total |
|---|---|---|---|---|
| Compound 10 | Putrescine | Spermidine | Spermine | Polyamines |
| Control | 7.1 ± 0.5 | 31.3 ± 0.3 | 18.3 ± 0.5 | 56.7 ± 0.3 |
| (0 μM) | | | | |
| 2 μM | 6.2 ± 0.7 | 27.1 ± 2.4 | 16.1 ± 0.0 | 49.5 ± 3.1 |
| 5 μM | 6.0 ± 0.9 | 17.6 ± 1.8 | 14.9 ± 1.1 | 38.5 ± 3.4 |
| 7 μM | 5.9 ± 0.3 | 15.6 ± 0.1 | 12.7 ± 0.6 | 34.2 ± 0.9 |

One way to affect both spermidine and spermine pools is to decrease methionine supply, which in turn would inhibit the formation of the decarboxylated S-adenosylmethionine needed to provide the aminopropyl fragment required for spermidine and spermine synthesis. Since methionine, leucine and phenylalanine all use the LAT1/SLC3A2 transporter to enter cells, it was hypothesized that 10 was inhibiting LAT-1 mediated amino acid import. To test this hypothesis, the development of various embodiments involved measuring the amino acid concentrations in the supernatants of cells incubated with compound 10 at 2 and 5 μM for 72 h. The results are shown in Table 2. It is maintained that other mechanisms may be involved in depletion of the amino acids in cells that do not involve importation.

Table 2. Concentration (pmoles/mg protein) of analytes collected in the supernatant of L3.6pl cell grown for 72 h at 37° C. in the presence of increasing doses of compound 10^{a}

TABLE 2A
Supernatant Levels in pmoles/mg protein

| | | | Acetyl | | Acetyl | Glutamic |
|---|---|---|---|---|---|---|
| Compd 10 | Putrescine | Spermidine | spermidine | Spermine | spermine | acid |
| 0 μM | 977 ± 522 | 339 ± 340 | 1936 ± 1365 | 6152 ± 5596 | 77 ± 43 | 23412 ± 3983 |
| 2 μM | 938 ± 70 | 131 ± 26 | 1649 ± 18 | 2532 ± 406 | 49 ± 10 | 20231 ± 649 |
| 5 μM | 2224 ± 98 | 167 ± 32 | 1706 ± 29 | 2690 ± 889 | 36 ± 4 | 34033 ± 540 |

TABLE 2B
Supernatant Levels in pmoles/mg protein

| Compd 10 | Agmatine | Arginine | Leucine | Methionine | Ornithine | Phenylalanine |
|---|---|---|---|---|---|---|
| 0 μM | 101 ± 142 | 22121 ± 10585 | 18009 ± 9061 | 22834 ± 1984 | 10588 ± 3212 | 14299 ± 7194 |
| 2 μM | 297 ± 163 | 18396 ± 70 | 15055 ± 1043 | 17686 ± 2432 | 9249 ± 306 | 11954 ± 828 |
| 5 μM | 485 ± 176 | 43243 ± 2047 | 202092 ± 23817 | 64036 ± 5454 | 15951 ± 118 | 160462 ± 18910 |

^{a} samples were run in duplicate.

As shown in Tables 2A and 2B3, significant increases in relative exogenous amino acid resources was observed in the presence of compound 10 (5 μM) as evidenced by higher levels of specific substrates outside the cells as measured in the media obtained from cells grown in the presence of compound 10 compared to the untreated control. The highest levels of exogenous amino acids were leucine (Leu), phenylalanine (Phe) and methionine (Met), i.e. the known LAT1 substrates. In short, there were high levels of LAT-1 substrates outside the cell after treatment with compound 10. In addition, the amount of Leucine and Methionine remaining inside cells after 72 h incubation with compound 10 was also determined. These results are shown in Table 3.

Table 3 shows intracellular concentrations after cell lysis (pmol/mg protein) after 72 h incubation of L3.6pl cells at 37° C. in the presence and absence of compound 10^{a}

TABLE 3
Intracellular levels

| Compound 10 | Leucine | Methionine |
|---|---|---|
| (μM) | (pmol/mg protein) | (pmol/mg protein) |
| 0 | 232 ± 10 | 167 ± 67 |
| 2 | 152 ± 8 | 226 ± 21 |
| 5 | 94 ± 3 | 41 ± 23 |
| 7 | 77 ± 1 | Not detected |

As shown in Table 3, a dose-dependent decrease in intracellular levels of both leucine and methionine were observed. Taken together, Tables 2A, 2B and 3 suggested that compound 10 affects large neutral amino acid pools and resulted in significant depletion of methionine and leucine pools inside these cells. Since large neutral amino acids utilize LAT proteins for cell entry, compound 10 may act as a direct or indirect LAT-1 (or LAT-2) inhibitor. Leucine and β-cyclohexylalanine (a reduced form of Phe) are used in the synthesis of 10. As a result, the molecular design of 10 (FIG. 3) contains isobutyl and cyclohexylmethyl substituents similar to the side chains of the natural substrates of LAT-1 (leucine and phenylalanine). According to various embodiments these features may provide 10 special affinity for the hydrophobic recognition sites on LAT-1.¹⁹ Its mechanism of action could involve direct LAT-1 inhibition to block uptake of LAT-1 substrates (e.g, methionine, leucine, and phenylalanine) and/or it could function by reversing the function of LAT-1 and exporting the LAT-1 substrates into the extracellular environment. This data suggests that these compounds likely act as LAT-1 uptake inhibitors and LAT-1 efflux agonists.

In parallel with the above experiments, a dose dependence experiment was performed looking at how compound 10 at 0, 2, 5 and 7 μM affected L3.6pl cell attachment and cell number after 72 h incubation at 37° C. Successively more detached cells were observed as the concentration of compound 10 increased, particularly from 5 μM to 7 μM. As shown in FIG. 9, a 58% and 80% loss of the attached cell population at 5 and 7 μM of compound 10 was observed, respectively, indicating that compound 10 is able to limit cell proliferation, presumably via polyamine depletion and nutrient starvation.

FIG. 9A is an example according to various embodiments, illustrating L3.6pl cells dosed with compound 10 at 0 μM. FIG. 9B is an example according to various embodiments, illustrating L3.6pl cells dosed with compound 10 at 2 μM. FIG. 9C is an example according to various embodiments, illustrating L3.6pl cells dosed with compound 10 at 5 μM. FIG. 9D is an example according to various embodiments, illustrating L3.6pl cells dosed with compound 10 at 7 μM.

Cells were incubated with compound 10 for 72 h at 37° C. A 250 μM AG solution was incubated with the cells for 24 h prior to compound addition. Each condition was performed in duplicate.

TABLE 4

| [Compd 10] | Cell Count | +/− Standard |
|---|---|---|
| μM | (millions) | deviation |
| 0 | 10.35 | 0.64 |
| 2 | 9.40 | 0.00 |
| 5 | 4.35 | 0.49 |
| 7 | 2.10 | 0.57 |

The 48 h IC₅₀ of compound 10 in L3.6pl cells was 3.48±0.30 μM. The 48 h IC₅₀ values for CHO K1 cells and CHO MG cells were 10.8±0.22 μM and 8.93±0.75 μM, respectively. The IC₅₀ values indicate that the compound is approximately three fold more toxic to L3.6pl cancer cells than to the CHO K1 and CHO-MG cell lines.

Prior experience with the ODC inhibitor (i.e., DFMO) demonstrated that DFMO-treated L3.6pl cells could recover their viability by replenishing their polyamine pools via spermidine import. 7 In addition, the AdoMetDC inhibitor (MDL73811) was shown to inhibit the growth of P. falciparum parasites and this growth inhibition was reversed by incubating infected erythrocytes with spermidine and spermine suggesting that cell treated with this inhibitor could also be rescued by exogenous polyamines.²⁰ In addition, prior work in L1210 murine leukemia cells demonstrated that inhibitors of AdoMetDC decrease intracellular spermidine and spermine levels, increase putrescine levels, and inhibit growth of L1210 cells.²¹ Addition of exogenous spermidine to L1210 cultures (containing the AdoMetDC inhibitor) was shown to restore normal growth rate.²¹ These observations suggest that AdoMetDC inhibition can, indeed, be overcome via polyamine import.

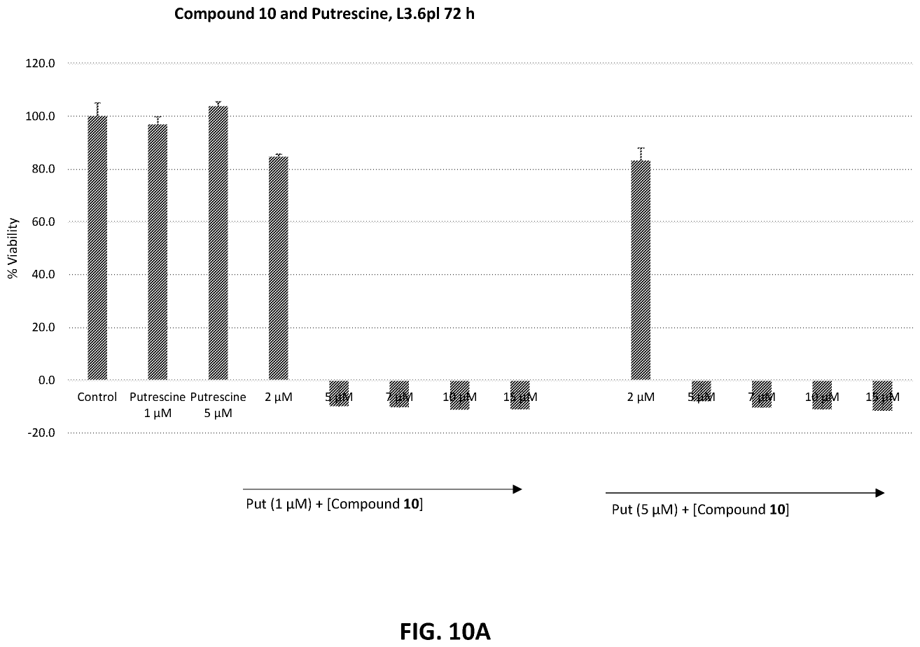

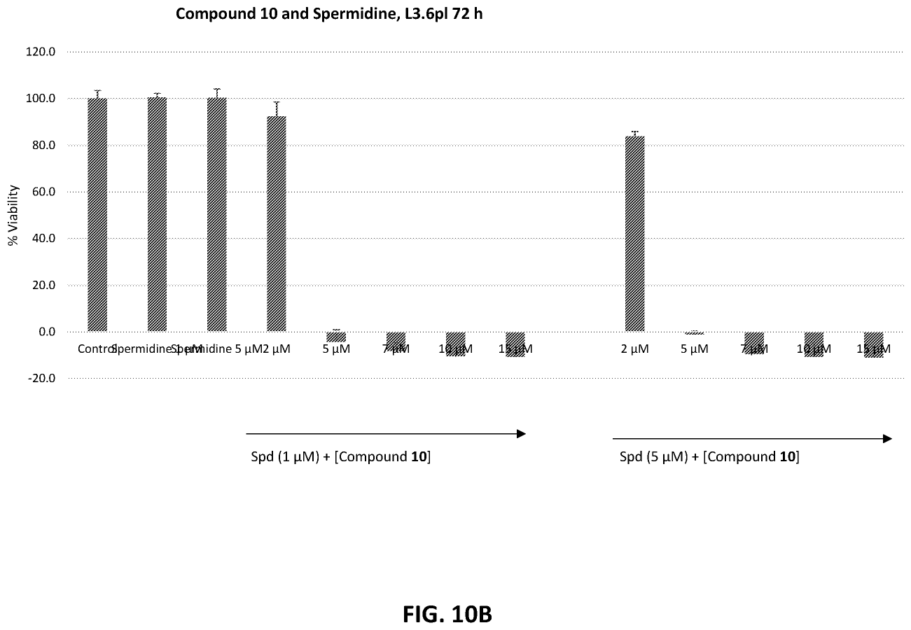

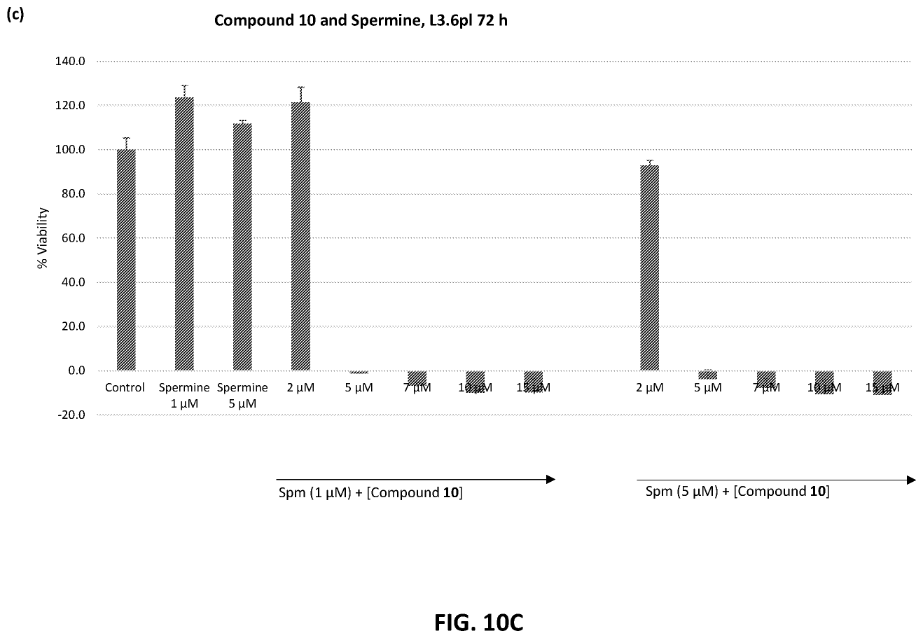

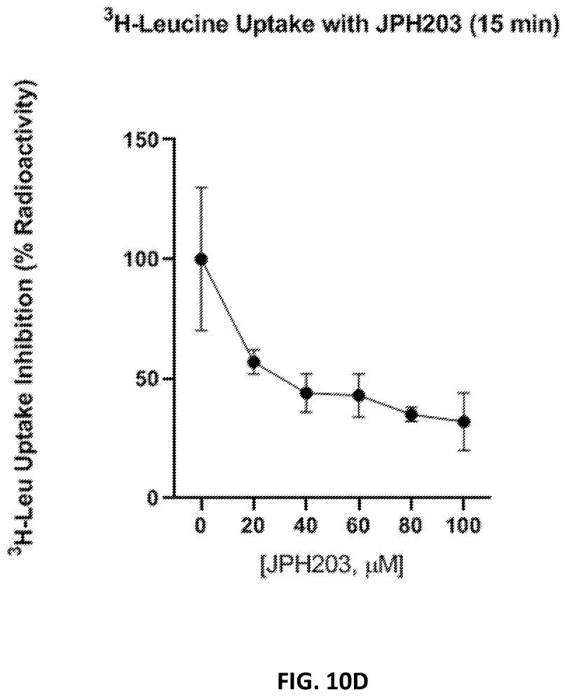

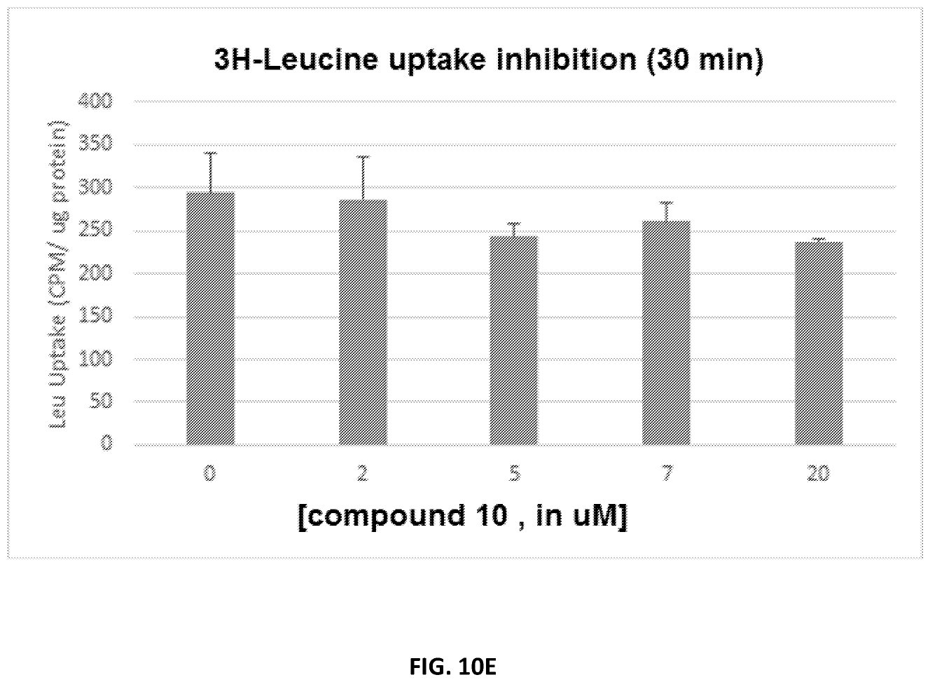

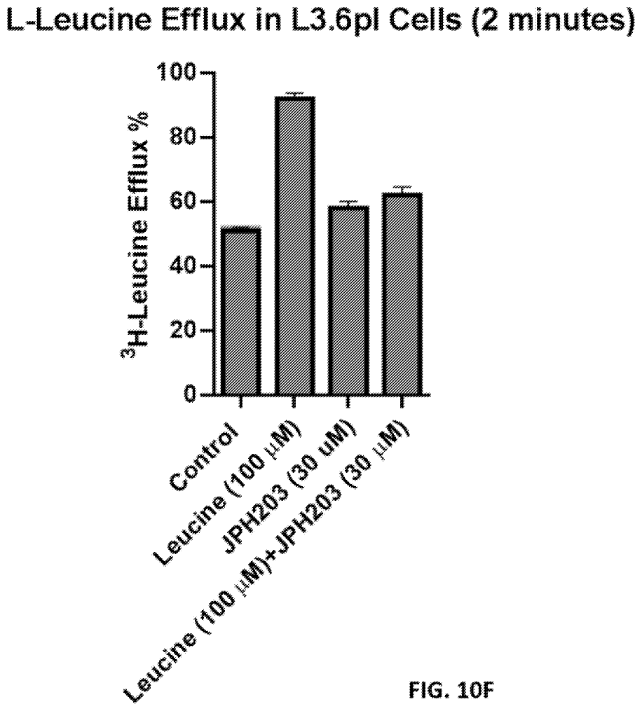

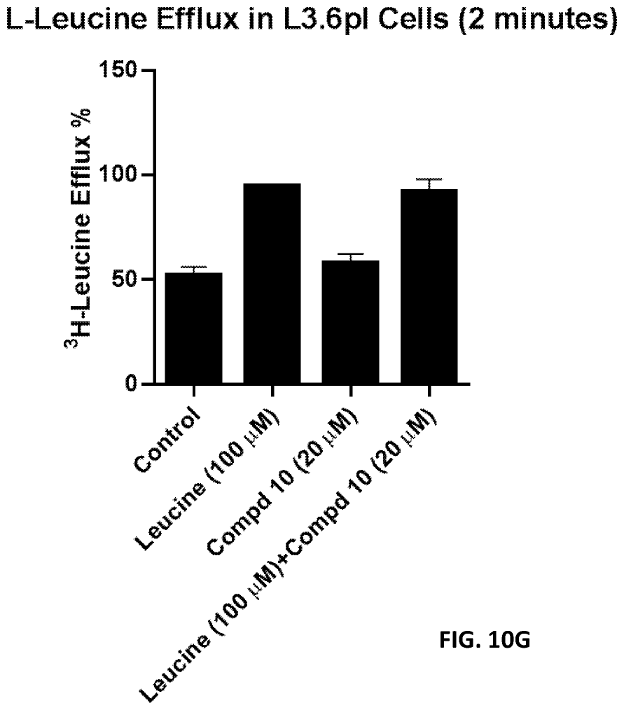

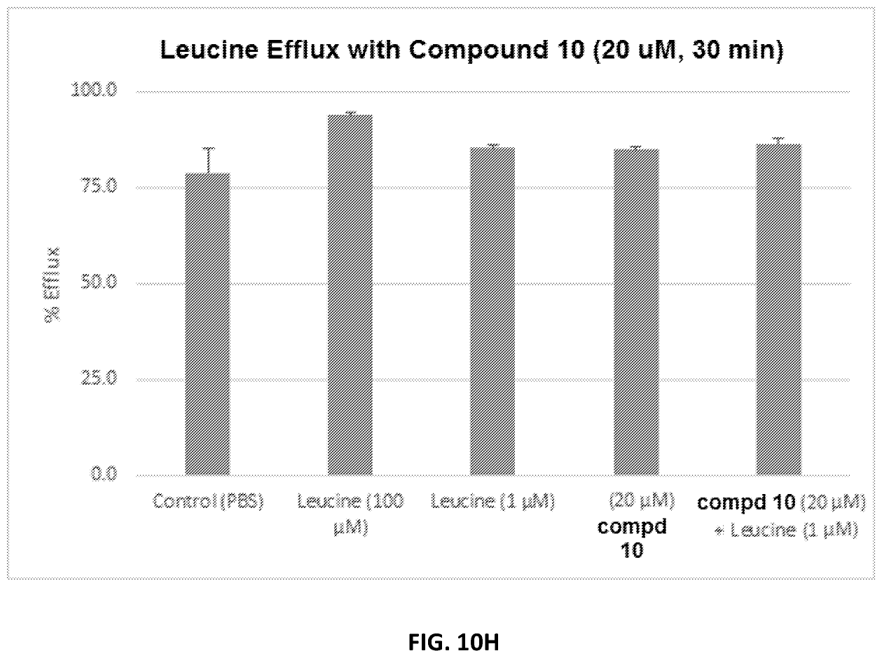

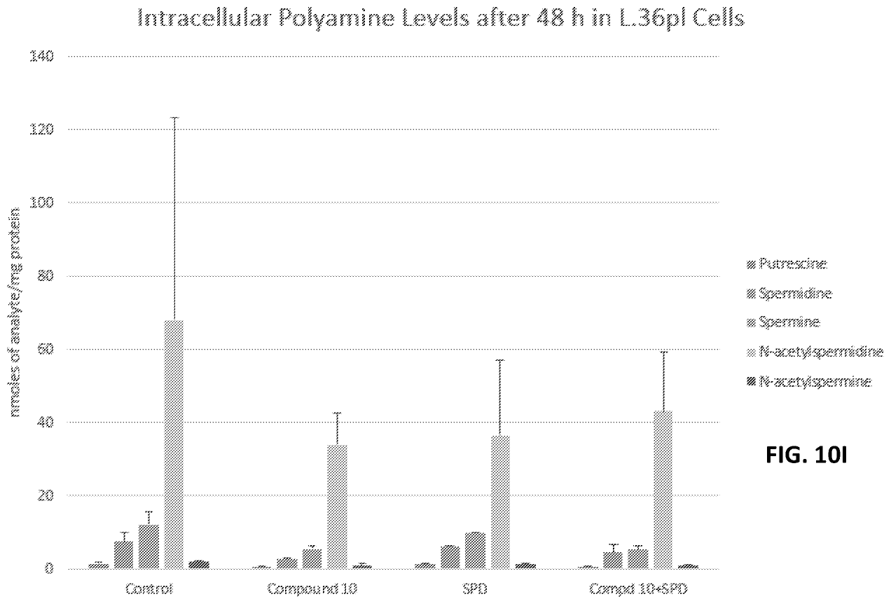

The data suggested that compound 10 causes a dose dependent decrease in intracellular polyamine levels. The question remained as to whether exogenous polyamines could enter and ‘rescue’ the cells back to the growth rate of untreated control. A series of experiments were conducted to determine if cells treated with compound 10 could be rescued by exogenous polyamines. To test this hypothesis, L3.6pl cells were incubated for 72 h with an increasing dose of compound 10 and a dose of one of the native polyamines (putrescine, spermidine, or spermine either at a fixed dose of 1 μM or 5 μM). The results are shown in FIG. 10. Unlike the observations made previously with DFMO, none of the three native polyamines (at 1 μM or 5 μM) were able to rescue L3.6pl cells treated with compound 10 (at a toxic dose, i.e. 5 μM 10 or higher). Similarly, the native polyamines were also unable to rescue CHO-K1 and CHO-MG cells treated with a toxic concentration of compound 10 (results not shown).

These collective results suggested that the polyamine depletion induced by 10 cannot be overcome by polyamine import. This was an important finding because cancer cells often escape inhibitors of the polyamine biosynthetic enzymes (e.g., DFMO) via polyamine import.^{7, 21} In short, inhibition of methionine supply provides a novel way to deplete intracellular spermidine and spermine pools without having to also inhibit polyamine import.²²

FIG. 10A is an example according to various embodiments, illustrating the inability of native polyamine putrescine (Put at 1 μM and 5 μM) to rescue L3.6pl cells treated with compound 10 (e.g., from 2-15 μM). FIG. 10B is an example according to various embodiments, illustrating inability of native polyamine spermidine (Spd at 1 μM and 5 μM) to rescue L3.6pl cells treated with compound 10 (e.g., from 2-15 μM). FIG. 10C is an example according to various embodiments, illustrating inability of the native polyamine spermine (Spm at 1 μM and 5 μM) to rescue L3.6pl cells treated with compound 10 (2-15 μM). The L3.6pl cells were incubated with 250 μM aminoguanidine (AG) for 24 h prior to the addition of compound 10, followed by 72 h incubation at 37° C. Columns 1-3 are control columns, with untreated L3.6pl pancreatic cancer cells as control and cells dosed with either 1 μM or 5 μM of the three native polyamines, respectively. Columns 4-8 and 9-13 show the results of experiments conducted with L3.6pl cells along with the respective native polyamine (fixed at either 1 or 5 μM) in the presence of increasing doses of 10. None of the three native polyamines were able to rescue L3.6pl cells treated with toxic doses of 10.

These studies indicated that the hit compound 10 identified from screening molecular libraries from the Torrey Pines Institute for Molecular Studies decreases intracellular leucine and methionine levels. The profound reduction of intracellular methionine pools led to significant reduction of intracellular spermidine and spermine pools in L3.6pl pancreatic cancer cells and inhibited cell growth.

As shown in FIG. 1, limited methionine supply has several consequences for the cell including a reduction in the decarboxylated S-adenosylmethionine pools needed to provide the aminopropyl fragments required to biosynthesize the higher polyamines (Spd and Spm). In this regard, compounds, which affect methionine supply, also impact polyamine homeostasis. Importantly, various embodiments show that the availability of exogenous native polyamines (Put, Spd or Spm) was not able to rescue cells treated with compound 10. This finding is in direct contrast to the ODC inhibitor (DFMO), where polyamine import provides an escape pathway for cancer cells to circumvent the ODC inhibitor. 7 In short, growth inhibitors like compound 10 may obviate the need for a PTI agent.

As shown in FIG. 1, since SLC3A2 (a.k.a. 4F2HC) has been shown in independent reports to associate with either LAT-1 (in T24 human bladder carcinoma cells)²³ or SAT1.²⁴ SAT1 (also known as SSAT) is a spermidine/spermine acetyl transferase which N-acetylates polyamines and facilitates their export. SLC3A2 may provide a molecular bridge for the coupling of neutral amino acid import and polyamine acetylation/export. The relative expression of LAT-1, SLC3A2, and SAT1 may therefore provide biomarkers for tumors most sensitive to this approach (i.e., treatment with compound 10). Tumors with low SLC3A2 expression may portray a tight regulation between amino acid import and polyamine export as both processes require SLC3A2. This regulation and balance between amino acid import/export and polyamine export will be particularly stressed in the presence of compounds which accelerate or block steps in the utilization of these resources such as a LAT-1 inhibitor, LAT-1 efflux agonist, or a SAT-1 inducer/agonist or a polyamine efflux agonist. Such agents increase the cell's demand for a particular transport pathway which requires SLC3A2.

Reduction in both methionine and leucine intracellular pools can explain the growth inhibition observed with compound 10. Indeed, leucine is an important signaling molecule for the mTOR pathway, which is known to control PDAC cell fate²⁵ and proliferation in PC-2 pancreatic carcinoma cells.²⁶ In this regard, decreasing the levels of LAT1 substrates (e.g., methionine and leucine) inside the cell may offer the opportunity to affect both polyamine metabolism and the mTOR pathway.

### Mechanism of Action Studies

FIG. 10D is an example according to various embodiments, illustrating dose dependent decrease in 3H-Leucine uptake (as measured in counts per minute (CPM)) observed in the presence of increasing concentration of the known LAT-1 inhibitor JPH-203. JPH203 is not toxic to L3.6pl cells over this concentration range and time interval. FIG. 10E is an example according to various embodiments, illustrating results obtained for a Leu uptake inhibition experiment with compound 10. Note: the y-axis in FIG. 10E is in CPM per ug of protein to normalize the data and account for any potential losses of cells due to toxicity of compound 10. FIG. 10F is an example according to various embodiments, illustrating results obtained for a Leucine efflux experiment with LAT-1 inhibitor JPH-203. Briefly, the efflux procedure involved cells pre-incubated with ‘hot’ leucine (3H labeled) and washed to remove unbound radiolabeled Leucine. The cells were then incubated in the presence and absence of unlabeled Leucine (100 μM) and/or the LAT-1 inhibitor JPH-203 (30 μM). In the presence of unlabeled leucine, the cells released ‘hot’ 3H-leucine into the media which was measured via scintillation/radioactivity counts. This efflux or release from within the cell was inhibited by the presence of the LAT-1 inhibitor, JPH-203 (30 μM, FIG. 10F). Since JPH203 inhibits the import of unlabeled leucine then less unlabeled Leucine will enter the cell and less efflux of radiolabeled Leucine molecules from inside the cell will be observed. This is consistent with JPH-203 being a LAT-1 inhibitor. FIG. 10G is an example according to various embodiments, illustrating results obtained for a two minute Leucine efflux experiment with compound 10. Cells were pre-incubated with ‘hot’ leucine (³H labeled) and washed to remove unbound radiolabeled Leucine. The cells were then incubated in the presence and absence of unlabeled “cold” Leucine (100 μM) and/or the compound 10 (20 μM). In the presence of unlabeled ‘cold’ leucine, the cells released ‘hot’ ³H-leucine into the media which was measured via scintillation/radioactivity counting. This cold leucine stimulated efflux or release of 3H-leucine from within the cell was not inhibited by compound 10 (20 μM) after 2 min. Indeed, a slight increase in efflux was observed with compound 10 alone at 20 μM. The efflux experiment was repeated and the time for monitoring efflux increased to 30 minutes. As shown in FIG. 10H, compound 10 by itself increased efflux of the radiolabeled leucine in the presence and absence of leucine (1 μM) compared to the untreated control. FIG. 10H provides evidence to suggest that compound 10 acts as a LAT-1 mediated export agonist, which effluxes LAT-1 substrates like methionine and leucine out of cells. This result/mechanism of action explains both the decrease in intracellular levels of these key amino acids and the observed increase in the supernatant of these amino acids. In summary, compound 10 inhibits Leucine uptake and facilitates LAT-1 mediated efflux.

An obvious way to decrease the levels of LAT-1 substrates inside the cell is to inhibit their import into the cell via LAT-1 inhibition. As shown in the latter panels of FIG. 10, it was demonstrated that the known LAT-1 inhibitor JPH-203 was able to block the uptake (FIG. 10D) of ³H-leucine in L3.6pl pancreatic cancer cells. JPH-203 also affected efflux (FIG. 10F), where it modestly stimulated efflux when dosed alone at 30 μM and was able to inhibit the larger efflux stimulation of 100 uM unlabeled Leucine (FIG. 10F) presumably by blocking the cellular entry of unlabeled leucine. In comparison, compound 10 was able to inhibit the uptake of 3H leucine (FIG. 10E) and also stimulated the efflux (FIG. 10G) of ³H-leucine in L3.6pl pancreatic cancer cells. Compound 10 at 20 μM (after 2 min) did not, however, inhibit the larger efflux stimulation of 100 μM unlabeled Leucine (FIG. 10G). As shown in FIG. 10H, compound 10 acts as a LAT-1 mediated amino acid export agonist as observed after a 30 minute incubation time (FIG. 10H) and to a lesser extent after 2 minutes of incubation (FIG. 10G). These experiments were consistent with each other and suggested that LAT-1 amino acid import inhibition as well as LAT-1 export agonism are involved in the mechanism of action for compound 10. This makes sense as an export agonist would also inhibit uptake processes through the same transport system.

Since compound 10 inhibits LAT-1 mediated import as measured by radiolabeled leucine import (See FIG. 10E), compound 10 also stimulates efflux (FIG. 10H). This result led us to speculate that compound 10 is involved in methionine efflux via the LAT-1 transporter working in reverse. This would explain the high levels of LAT-1 substrates (methionine, leucine and phenylalanine) outside the cell in cells treated with compound 10 (Table 2B). This would arise not from the inhibition of import of these substrates, but instead via the directed efflux of these amino acids to outside the cell. This phenomenon is possible as LAT-1 is a natural antiporter, where intracellular glutamine is exchanged for extracellular large neutral amino acids (e.g., leucine, methionine). Using a two-minute observation period, enhanced efflux by compound 10 (FIG. 10G) was only modestly observed. Repeating this experiment for a longer incubation period (30 minutes) also demonstrated compound 10's agonism of leucine export.

Beyond compound 10 inducing LAT-1 mediated efflux, there are other potential mechanisms of action to explain methionine depletion by compound 10 that cannot be ruled out at this time. While LAT-1 is the likely target, it is possible that compound 10 also works by stimulating increased metabolic flux through methionine-dependent pathways, which consume the available intracellular methionine pools. Typically, methionine is converted to S-adenosylmethionine (SAM) to provide carbon sources for many cellular processes. For example, agonism of intracellular methylation processes, which consume SAM resources, would also deplete methionine pools. As recently shown in yeast, SAM is consumed by the methyltransferase CHO2 during the methylation of phosphatidylethanolamine (PE) for the synthesis of phosphatidylcholine (PC). These ubiquitous membrane components (PE and PC) provide a large ‘methyl sink’ for SAM. Compounds which act as CHO2 agonists could accelerate this process and lead to SAM and methionine depletion. Another example is the use of SAM for the methylation of nicotinamide via nicotinamide N-methyltransferase (NNMT). Indeed, nicotinamide N-methyltransferase (NNMT) can regulate histone methylation by changing the intracellular levels of SAM. NNMT agonists would consume SAM pools and result in methionine depletion. There are many other potential examples of methionine donating methyl groups via its SAM metabolite, including DNA and histone-methylation pathways. Again, agonism of these and other methionine dependent pathways would consume SAM pools and offer alternative explanations for the mechanism of action of compound 10.

While methionine is critical for one carbon metabolism and methyl transfer reactions, SAM can also be a source for the transfer of aminopropyl groups. In a different pathway, SAM is decarboxylated to form decarboxylated-SAM, which donates an aminopropyl group to build the higher polyamines spermidine and spermine using the two biosynthetic enzymes, spermidine synthase (SRM) and spermine synthase (SMS), respectively. Therefore, polyamine biosynthesis also consumes SAM pools in forming spermidine and spermine. Indeed, agonism of polyamine efflux is another potential mechanism to explain how compound 10 may function. A dose dependent decrease in polyamine pools was observed in the presence of compound 10 (FIG. 8). This could be explained by the lack of methionine to make these polyamines. Alternatively, if polyamine efflux was stimulated by compound 10, then intracellular polyamines would be N-acetylated and exported into the extracellular space and could explain the downward trend in total polyamine levels observed in FIG. 8. Without wishing to be bound by theory, it is speculated that if the polyamine efflux rate were high enough, then the polyamine ‘bleed rate’ would be faster than the methionine dependent polyamine biosynthetic process resulting in a futile consumption of SAM to make spermidine and spermine, which are subsequently exported. This SAM-driven futile effort results in a net loss of polyamines and methionine (as observed). To explore this possibility, polyamine levels inside L3.6pl cell treated with 3.3 μM of compound 10 (SR isomer) were measured. As shown in FIG. 10i, there are, indeed, lower levels of polyamines and N-acetylspermine inside L3.6pl cells treated with compound 10 (consistent with FIG. 8). If this mechanism were in play, however, one should see a build-up of acetylated polyamines outside the cell. As shown in Table 2A, there is not a pronounced increase in acetylated polyamines in the supernatants of L3.6pl cells treated with compound 10. This observation, therefore, ruled out compound 10 acting as a polyamine efflux agonist.

In summary, while the precise mechanism of action of compound 10 is not yet defined, the most likely mechanism that fits the data is one of LAT-1 mediated transport, where the compound causes LAT-1 to export large amino-acids like methionine, leucine, and phenylalanine into the extracellular space and inhibits the uptake of exogenous LAT-1 substrates as evidenced in Table 2B and FIG. 10H. However, inhibition of importation is not a limiting theory of mechanism of action, and other mechanism may be responsible for the depletion of amino acids in the cells.

### EXAMPLES

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. The purpose of the following examples is not to limit the scope of the various embodiments, but merely to provide examples illustrating specific embodiments.

A variety of materials were used to perform the following examples. Silica gel 32-63 μm and chemical reagents were purchased from commercial sources and used without further purification. ¹H and ¹³C spectra were recorded at 400 MHz and 100 MHz, respectively. NH₄OH referred to concentrated aqueous ammonium hydroxide. All tested compounds provided satisfactory elemental analyses as proof of purity (≥95%). These are provided in the Supporting Information.

Regarding the biological studies performed in the following examples, it is noted that CHO K1, CHO-MG, and L3.6pl cells were grown in RPMI 1640 medium supplemented with 10% fetal bovine serum (FBS) and 1% penicillin/streptomycin. All cells were grown at 37° C. under a humidified 5% CO₂ atmosphere. Aminoguanidine (1 mM used for CHO K1 and CHOMG cells and 250 μM for L3.6pl) was added to the growth medium to prevent oxidation of the compounds of the bovine serum amine oxidase enzyme that is present in calf serum. The cells used were in early to mid log phase and CHO and CHOMG were plated out at 1000 cells/well, whereas the L3.6pl cells were plated at 500 cells/well in a 96 well plate format.

### Example 1

This example illustrates aspects according to various embodiments pertaining to IC₅₀ determinations and cell viability studies. Cell growth was assayed in sterile 96-well microtiter plates (Costar 3599, Corning, N.Y.). CHO K1 or CHOMG cells were plated at 1,000 cells/70 μL and L3.6pl cells at 500 cells/70 μL. The drug solutions of appropriate concentration in phosphate buffered saline (PBS) were added 10 μL per well after overnight incubation. After drug exposure (e.g., for 48 h for CHO K1 and CHO-MG and 48 h or 72 h for L3.6pl), cell growth was determined by measuring formazan formation from the 3-(4,5dimethylthiazol-2-yl)-5-(3-carboxymethoxyphenyl)-2-(4-sulfenyl)-2H tetrazolium, inner salt (MTS) using SynergyMx Biotek microplate reader for absorbance (490 nm) measurements.²⁷ All experiments were run in triplicate.

### Example 2

This example illustrates aspects according to various embodiments pertaining to a 72 h experiment with Compound 10 in L3.6pl cells. Ten milliliters of a cell suspension containing L3.6pl human pancreatic cancer cells (50,000 cells/mL) and aminoguanidine (1 mM) were placed in treated plastic Petri dishes (d=9 cm) and incubated overnight at 37° C. After 24 h, the cells were dosed with compound 10 at 2 μM, 5 μM, or 7 μM. (Note: Stock solutions of 10 at 2, 5, and 7 mM were dissolved in phosphate buffered saline (PBS) and were filtered through a 0.2 μm filter prior to use. The experiments were maintained with a total volume of 10.01 mL where 10 mL of the cell suspension was placed in a plastic dish and each compound or the equivalent PBS volume was added to make up the total volume 10.01 mL for each dish. For example, for the control experiment PBS (10 μL) was added to make up the total volume (10 mL+10 μL=10.01 mL). After 72 h incubation, the cells were collected first by pipetting off the supernatant containing floating dead cells from the culture dish as well as media and placing them into 15 mL tubes. The supernatant containing floating cells was centrifuged (4 min at 1,000 rpm). The cell-free supernatant (supernatant #1) was collected into a new 15 mL tube and quantified (˜8.6 mL) and was then stored frozen and was later quantified by LCMS to investigate the media composition of particular polyamine and amino acid analytes. The attached cells on the dish were washed with PBS (5 mL). The PBS wash was removed by suction and additional PBS (2 mL) was added and again suctioned off to provide twice-washed cells still adhered to the dish. Trypsin (2 mL) was then added to each dish and incubated (3-5 min) until all the cells were detached. Fresh media (8 mL) was added to quench the trypsin. The cell solutions were pipetted into separate 15 mL tubes and centrifuged (4 min at 1,000 rpm). The resulting supernatant was removed to provide a pellet. The pellet was suspended in PBS (10 mL) and was counted by a hemocytometer to provide cell counts for each experimental condition. The pellet was then centrifuged (4 min at 1,000 rpm) and the supernatant removed and the remaining pellet was quantified via protein and polyamine analysis.

To each cell pellet, a perchloric acid (100 μL) buffer solution (0.2M HClO₄/1 M NaCl) and 0.9% NaCl (50 μL) was added. The samples were sonicated via sonic dismembranator in small bursts until samples were homogenized and cloudy. Additional perchloric acid (50 μL) buffer solution was added. The homogenized samples were then vortexed and centrifuged (10 min at 4,000 rpm). The supernatants of the respective samples (supernatant #2) were removed and quantified by calibrated pipet (˜190 μL volume). Note: 100 μL of supernatant #2 was placed into a micro-centrifuge tube for polyamine analysis by the N-dansylation HPLC protocol and the rest was placed into a different micro-centrifuge tube for analysis by LCMS of specific amino acid analytes. The respective supernatants were stored in the freezer for polyamine and LCMS quantification and the remaining protein pellet was used for the protein analysis. The protein pellet was dissolved in aq. NaOH (1 mM, 200 μL). The sample stood at room temperature with occasional vortex (45 min) and was then centrifuged (15 min at 15,000 rpm). The supernatant was collected and dissolved protein was quantified using the commercial Pierce BCA kit according to manufacturer's protocol.

### Example 3

This example illustrates aspects according to various embodiments pertaining to a polyamine analysis protocol via N-dansylation and HPLC. Internal standard (1,7-diaminoheptane at 1.5×10⁻⁴ M) was added (30 μL) to supernatant #2 (100 μL sample) as well as 1 M aqueous sodium carbonate solution (200 μL) and dansyl chloride (5 mg/mL) in acetone solution (400 μL). The sample mixture was vortexed and was then placed on a rotary shaker (65° C. for 60 min at 200 rpm). Proline solution (1 M, 100 μL) was then added and the sample was placed on a rotary shaker (65° C. for 20 min at 200 rpm). The solution was transferred to a glass vial. Chloroform (1 mL) was added and the vial was vigorously shaken and placed on counter to allow the layers to separate and the top aqueous layer was removed. The sample was concentrated under reduced pressure using a rotary evaporator. Methanol was added (1 mL) to dissolve the remaining residue in the glass vial. Samples were filtered via C18 filtered cartridge (Thermo Scientific hypersep C18, 50 mg bed weight) and the cartridge was pre-wetted with methanol (1 mL) and the liquid was pushed through with nitrogen gas. The sample dissolved in methanol was then added to the top of the cartridge and forced through with nitrogen gas and then additional methanol (0.5 mL) was added and flushed through to collect the sample in a HPLC vial and polyamine analysis was performed via HPLC using gradient elution of acetonitrile and a heptanesulfonate aqueous buffer.²⁸

### Example 4

This example illustrates aspects according to various embodiments pertaining to a protocol for polyamine level determination in FIGS. 7 and 8. L3.6pl cells (500,00 cells/10 mL media) were incubated with aminoguanidine (250 μM) at 37° C. for 24 h. Each compound was then added either alone or in combination with other agents (e.g., Ant44, 10 μL of appropriate stock solution) as indicated in FIGS. 7 and 8. The total volume in each dish was kept constant via the addition of PBS when needed, and the cells were incubated for another 72 h at 37° C. The cells were then washed extensively with ice cold PBS (once with 5 mL and twice with 2 mL). Each PBS wash was removed by suction. To the washed cells, an additional 2 mL of ice cold PBS was added and the cells were scraped off the dish and collected in a centrifuge tube. The cell suspensions were then centrifuged at 1,000 rpm for 4 minutes to provide a cell pellet. The supernatant was carefully removed by suction. The cell pellet was lysed using a 0.2 M perchloric acid/1 M NaCl solution (200 μL), sonicated, and centrifuged. The resultant supernatant and pellet were separated. The supernatant volume was measured via calibrated pipet (˜190 μL) and then used to quantify the respective N-dansylated polyamines by derivatization and HPLC analysis as described above.²⁸ The protein content of the pellet was quantified using the Pierce BCA Protein assay kit from Thermo Scientific. Final results were expressed as nmol polyamine/mg protein. Each condition was performed in duplicate.

### Example 5

This example illustrates aspects according to various embodiments pertaining to an LCMS Analysis. The respective supernatant (10 μL) was injected on a Thermo HPLC system equipped with PAL CTC plate sampler (96-well plate), Dionex Ultimate 3000 binary pump (flow rate at 0.25 mL/min), Dionex Ultimate 3000 thermostatted column compartment (temperature at 40° C.), Thermo Endura Mass Spectrometer (ESI source), using Thermo Scientific Accucore C18 (2.6 μm, 2.1×50 mm, 100 Å) column under a gradient of acetonitrile w/0.1% heptafluorobutyric acid (HFBA) in H₂O w/0.1% HFBA from 2% at minute 0 to 60% at minute 5.0, to 99% at minute 6.5 held until minute 7.5 and then reduced back to 2% until minute 10 to re-equilibrate the column for the next injection. The peak area was measured and analyte amounts were calculated referring to analyte calibration curves. Analyte levels were adjusted with internal standard concentration for extraction efficiency. Peak height measurements were conducted referring to values obtained for standards of known concentrations. Calibration curves were constructed from eight concentrations (1, 5, 10, 50, 100, 500, 1000 and 5000 nM) by spiking 10 μL of 50× concentration DMSO stocks into 490 μL buffer and extracting 25 μL of the resulting sample and analyzing as detailed above. The LCMS data were originally reported in nM and then converted to pmoles analyte/mg protein by multiplying by the respective supernatant volume collected (e.g., supernatant #1, ˜8.6 mL; supernatant #2, ˜190 μL) and dividing by the mg of protein determined for the cell pellet by the BCA method obtained for that particular supernatant #1 and supernatant #2 sample. In this manner, the data for both the extracellular and intracellular analytes were expressed in the same pmol/mg protein units and are listed in the respective Tables.

### Example 6

This example demonstrates the synthesis of (S)-2-(3,3-Dimethyl-butyrylamino)-3-phenyl-propionic acid methyl ester (14, 177-1). To the solution of 3,3-dimethylbutryic acid 12 (1.1 mL, 8.61 mmol, 1 equiv) and L-phenylalanine methyl ester hydrochloride 13 (1.86 g, 8.61 mmol, 1 equiv) in DCM (40 mL) was added diisopropylethylamine (3.01 mL, 17.2 mmol, 2 equiv) followed by HATU (6.55 g, 17.2 mmol, 2 equiv) and stirred for 24 hrs at room temperature. The reddish brown reaction mixture turned milky white overnight. The reaction mixture was quenched by washing with aqueous Na₂CO₃, followed by extraction with DCM. This organic layer was then washed with water, dried over anhydrous Na₂SO₄, filtered and concentrated. The crude product was purified by flash column chromatography (100% CHCl₃) to give the pure coupled product 14 (177-1) as a yellow oil (95%). ¹H NMR (500 MHz, CDCl₃): δ 7.31-7.22 (m, 3H), 7.11 (m, 2H), 5.76 (br s, 1H), 4.91 (m, 1H), 3.72 (s, 3H), 3.11 (m, 2H), 2.04 (s, 2H), 0.98 (s, 9H). ¹³C NMR (500 MHz, CDCl₃): δ 172.2, 171.3, 135.9, 129.2, 128.6, 127.1, 52.9, 52.2, 50.3, 38.0, 30.9, 29.7. HRMS m/z calc for C₁₆H₂₃NO₃ (M+H)⁺ theory: 277.1678, found: 277.1653. Anal. Chem. C₁₆H₂₃NO₃, CHN. (i.e., the compound passed elemental analysis and is >95% pure).

### Example 7

This example demonstrates the synthesis of 2-(3,3-Dimethyl-butyrylamino)-3-phenyl-propionic acid (15, 177-2). 1 M NaOH (7.6 mL, 7.6 mmol) was added slowly to methyl ester 14 (177-1) (2.1 g, 7.57 mmol) in MeOH (75 mL) at 0° C. with stirring. The mixture was allowed to warm to room temperature and stir until consumption of the methyl ester was observed by TLC (1% MeOH in DCM). After 24 hours, the reaction mixture was concentrated under reduced pressure. The resulting oil was then cooled to 0° C. taken up in 130 mL of 0.1 M HCl. A white precipitate with limited solubility formed during HCl addition. Vacuum filtration using cold DCM was used to isolate the solid. The liquid filtrate was extracted two times with ethyl acetate and the combined organic layers were dried over anhydrous Na₂SO₄, filtered, and concentrated to increase yield. ¹H NMR (CDCl₃) was used to confirm the loss of the methyl ester singlet (3.73 ppm). The carboxylic acid product 15 (177-2) (1.976 g, 99%) was consumed in the next step. ¹H NMR (500 MHz, CDCl₃): δ 7.33-7.24 (m, 3H), 7.19 (m, 2H), 5.74 (m, 1H), 4.84 (td, 1H, J³_{H-H}=7.2 Hz, 7.2 Hz, 5.6 Hz), 3.19 (m, 2H), 2.05 (m, 2H), 0.94 (s, 9H). HRMS m/z calc for C₁₅H₂₁NO₃ (M+H)⁺ theory: 263.1521, found: 263.1547. Anal. Chem. C₁₅H₂₁NO₃, CHN.

### Example 8

This example demonstrates the synthesis of N-[1-(1-Carbamoyl-2-phenyl-ethylcarbamoyl)-2-phenyl-ethyl]-3,3-dimethyl-butyramide (compound 17, 177-3). To the solution of 2-(3,3-Dimethyl-butyrylamino)-3-phenyl-propionic acid 15 (177-2) (773 mg, 2.93 mmol, 1 equiv) and L-phenylalanine amide 16 (488 mg, 2.97 mmol, 1 equiv) in DCM (25 mL) was added diisopropylethylamine (1.02 mL, 5.87 mmol, 2 equiv), followed by HATU (2.34 g, 6.15 mmol, 2 equiv) and stirred for 3 days at room temperature. A white precipitate formed over the course of the reaction. The reaction was filtered and the resulting precipitate was taken up in hot ethyl acetate and washed with 0.1 M HCl, aqueous Na₂CO₃, and water. The organic layer was dried over anhydrous Na₂SO₄, filtered, and concentrated to yield the pure triamide 17 (177-3) as a white solid (464 mg, 39%). ¹H NMR (500 MHz, d₆-DMSO): δ 7.90 (m, 2H), 7.34-7.05 (m, 12H), 4.51 (m, 1H), 4.43 (m, 1H), 2.98 (m, 2H), 2.84 (m, 1H), 2.68 (m, 1H), 1.89 (s, 2H), 0.77 (s, 9H). ¹³C NMR (500 MHz, d₆-DMSO): δ 172.6, 171.2, 170.8, 138.0, 137.7, 129.2, 129.1, 128.0, 127.9, 126.2, 126.1, 53.8, 53.6, 48.5, 37.6, 37.2, 30.3, 29.5. HRMS m/z calc for C₂₄H₃₁N₃O₃ (M+H)⁺ theory: 409.2365, found: 409.2386. Anal. Chem. C₂₄H₃₁N₃O₃, CHN.

### Example 9

This example demonstrates the synthesis of N²-[2-(3,3-Dimethyl-butylamino)-3-phenyl-propyl]-3-phenyl-propane-1,2-diamine hydrochloride salt (18, 177-4). Borane-tetrahydrofuran complex (1.0 M, 13.7 mmol, 8 equiv., 13.7 mL) was added via a syringe to N-[1-(1-Carbamoyl-2-phenyl-ethylcarbamoyl)-2-phenyl-ethyl]-3,3-dimethyl-butyramide 17 (177-3) (700 mg, 1.71 mmol, 1 equiv) in THF (43 mL) at ambient temperature. The mixture was then heated at 60-65° C. After refluxing for 4 days, the reaction mixture was concentrated under reduced pressure to give a residue. A 10% concentrated HCl/Methanol solution (30 mL) was then added at 0° C. and stirred for 24 hrs. The mixture was concentrated to give a residue, which was taken up in 1 M NaOH until reaching pH 10, then extracted three times with DCM, dried over Na₂SO₄, filtered, and concentrated. The crude triamine free base (610 mg) was purified by flash column chromatography (5% MeOH, 1% NH₄OH in DCM) to give the pure triamine 18 (177-4) as a yellow oil (330 mg, 52%). ¹H NMR (500 MHz, CDCl₃): δ 7.20 (m, 4H), 7.13 (m, 2H), 7.05 (m, 4H), 2.74 (m, 2H), 2.70-2.34 (m, 10H), 1.23 (m, 2H), 0.79 (s, 9H). ¹³C NMR (500 MHz, CDCl₃): δ 139.12, 139.06, 129.01, 128.98, 128.14, 128.12, 125.9, 125.8, 61.3, 59.4, 53.3, 49.3, 44.3, 43.9, 42.9, 38.8, 29.3.

### Example 10

This example demonstrates the synthesis of N-{2-[2-(3,3-Dimethyl-butylamino)-3-phenyl-propylamino]-3-phenyl-propyl}-benzamide (20, 177-5). A solution of N-(benzoyloxy)succinimide 19 (212.6 mg, 0.97 mmol, 1 equiv) in DCM (2 mL) was added dropwise to a stirred solution of triamine 18 (177-4) (358 mg, 0.97 mmol, 1 equiv) in DCM (2 mL) at 0° C. The reaction mixture was allowed to warm to room temperature and stir until TLC (7% MeOH, 1% NH₄OH in DCM) showed complete consumption of the starting material. After 19 hours, the reaction mixture was washed with aqueous Na₂CO₃, dried over anhydrous Na₂SO₄, filtered, and concentrated to give a crude residue. The crude residue was purified by flash column chromatography (3% MeOH, 1% NH₄OH in DCM) to give pure 20 (177-5) as an oil (412 mg, 90%). ¹H NMR (500 MHz, CDCl₃): δ 7.73 (m, 2H), 7.41 (m, 1H), 7.34 (m, 2H), 7.26-7.07 (m, 12H), 7.00 (m, 2H), 3.47 (dt, 1H, J²_{H-H}=13.6 Hz, J³_{H-H}=4.6 Hz, 4.6 Hz), 3.32 (dt, 1H, J²_{H-H}=13.6 Hz, J³_{H-H}=6.0 Hz, 6.0 Hz), 2.97 (m, 1H), 2.81-2.35 (m, 10H), 1.11 (m, 2H). ¹³C NMR (500 MHz, CDCl₃): δ 167.6, 138.6, 138.3, 134.7, 131.3, 129.2, 129.1, 128.7, 128.54, 128.45, 127.1, 127.0, 126.9, 126.6, 126.4, 59.4, 58.5, 48.4, 43.7, 42.9, 42.4, 39.4, 38.6, 29.7, 29.6, 29.52, 29.45. HRMS m/z calc for C₃₁H₄₁N₃O (M+H)⁺ theory: 471.3250, found: 471.3211. Anal. Chem. C₃₁H₄₁N₃O, CHN.

### Example 11

This example demonstrates the synthesis of N-{2-[5-Benzyl-4-(3,3-dimethyl-butyl)-2,3-dioxo-piperazin-1-yl]-3-phenyl-propyl}-benzamide (21, 177-6). To a solution of diamine 20 (177-5) (0.02 M, 197 mg, 0.418 mmol, 1 equiv) in DCM (10 mL) at 0° C. was slowly added a 5-fold excess of oxalyldiimidazole (0.1 M, 397 mg, 2.09 mmol, 5 equiv) in DCM (11 mL). The resulting reaction mixture was allowed to stir at room temperature 3 hours and monitored by TLC (7% MeOH, 1% NH₄OH in DCM). The reaction mixture was concentrated under reduced pressure after 3 hrs. The crude reaction residue (603 mg) was purified by flash column chromatography (2% MeOH in DCM) to give the cyclized product 21 (177-6) with enhanced purity (192 mg). An impurity was still observed by NMR so a second column was performed (40% EtOAc, 1.5% EtOH in hexanes) to give the pure cyclized product 21 (177-6) as a white powder (173 mg, 79%). ¹H NMR (500 MHz, CDCl₃): δ 7.78 (d, 1H, J³_{H-H}=7.3 Hz), 7.40 (m, 1H), 7.32 (m, 2H), 7.24-7.10 (m, 9H), 6.80 (d, 2H, J³_{H-H}=7.3 Hz), 4.58 (br s, 1H), 3.92 (m, 1H), 3.58 (m, 3H), 3.37 (m, 1H), 3.16 (m, 1H), 3.06 (d, 1H, J²_{H-H}=13.2 Hz), 2.94 (d, 1H, J³_{H-H}=6.6 Hz) 2.56 (m, 1H), 2.44 (m, 2H), 1.29 (td, 1H, J²_{H-H}=12.3 Hz, 12.3 Hz), 1.18 (m, 2H), 0.73 (s, 9H). ¹³C NMR (500 MHz, CDCl₃): δ 208.8, 205.4, 166.9, 158.0, 155.6, 140.7, 135.6, 132.7, 130.5, 127.98, 127.95, 127.90, 127.87, 127.5, 126.19, 126.16, 126.1, 55.3, 42.5, 41.0, 39.8, 36.8, 35.0, 28.7, 28.1. HRMS m/z calc for C₃₃H₃₉N₃O₃ (M+H)⁺ theory: 525.2991, found: 525.2991. Anal. Chem. C₃₃H₃₉N₃O₃, CHN.

### Example 12

This example demonstrates the synthesis of Benzyl-{2-[3-benzyl-4-(3,3-dimethyl-butyl)-piperazin-1-yl]-3-phenyl-propyl}-amine trihydrochloride salt (9, 1666.177). 1.98 mL of Borane-tetrahydrofuran complex (1.0 M, 1.98 mmol, 8 equiv) was added via a syringe to 21 (177-6) (130 mg, 0.247 mmol, 1 equiv) in THF (3 mL) at ambient temperature. The mixture was then heated at 60-65° C. for 2 days. The reaction mixture was concentrated under reduced pressure to give a residue. A 10% concentrated HCl/Methanol solution (4 mL) was then added at 0° C. and stirred for 24 hrs. The mixture was concentrated to give a residue, which was taken up in 1 M NaOH until reaching pH 10, then extracted three times with DCM, dried over anhydrous Na₂SO₄, filtered, and concentrated. The crude triamine free base (107 mg) was purified by flash column chromatography (3% MeOH, 1% NH₄OH in DCM) to give the pure free base of 9 (1666.177) as a yellow oil (77.8 mg, 65%). A portion of the free base of 1666.177 (45 mg) was dissolved in absolute ethanol (3 mL) at 0° C. A 4 N HCl solution (6 mL) was slowly added to the free base solution. The solution was stirred for 30 minutes then concentrated. The resulting white solid was then taken up in water and concentrated to remove any remaining ethanol, giving the respective amine HCl salt of 9 (1666.177) (51.8 mg) as a crystalline solid. ¹H NMR (400 MHz, D20): δ 7.50-7.05 (m, 15H), 4.12 (m, 2H), 3.58-2.57 (m, 13H), 2.55-2.38 (m, 2H), 1.80-1.46 (m, 1H), 0.96 (s, 9H). ¹³C NMR (100 MHz, D20): δ 139.3, 136.5, 131.9, 131.6, 131.5, 131.0, 130.8, 130.7, 130.6, 129.3, 128.6, 64.1, 63.2, 55.4, 52.9, 52.0, 51.8, 46.3, 42.9, 37.2, 35.8, 33.5, 30.8, 29.8.

### Example 13

This example demonstrates the synthesis of 4-Methyl-2-(3-methyl-butyrylamino)-pentanoic acid ethyl ester (24, 255-1). A procedure similar to that described above for 14 was used to prepare 24 (255-1) using isovaleric acid 22 and L-leucine ethyl ester hydrochloride 23. After 24 h, the TLC (2% MeOH in DCM) showed disappearance of the starting material. The reaction mixture was quenched by washing with aqueous Na₂CO₃, followed by extraction with DCM. The organic layer was collected and washed with 0.01 M HCl. The resulting organic layer was collected and washed with water, dried over anhydrous Na₂SO₄, filtered, and concentrated. The crude was purified through the flash column chromatography (DCM to 1% MeOH in DCM). However, the product coeluted with the urea by-product. A second column was done using 0.5% MeOH in DCM to give the pure coupled product 24 as a white solid (93%). ¹H NMR (500 MHz, CDCl₃): δ 5.93 (d, 1H, J³_{H-H}=8.1 Hz), 4.64 (td, 1H, J³_{H-H}=8.7 Hz×2), 4.18 (q, 2H, J³_{H-H}=7.3 Hz×3), 2.17-2.06 (m, 3H), 1.66 (m, 2H), 1.54 (m, 1H), 1.28 (t, 3H, J³_{H-H}=7.2 Hz×2), 0.95 (m, 11H). ¹³C NMR (500 MHz, CDCl₃): δ 173.2, 172.2, 61.2, 50.5, 45.9, 41.7, 26.1, 24.8, 22.8, 22.4, 21.9, 14.1. HRMS m/z calc for C₁₃H₂₅NO₃ (M+H)⁺ theory: 244.1907, found: 244.1909. Anal. Chem. C₁₃H₂₅NO₃, CHN.

### Example 14

This example demonstrates the synthesis of 4-Methyl-2-(3-methyl-butyrylamino)-pentanoic acid (25, 255-2). Aqueous NaOH (1 M, 3 mL, 3 mmol) was added slowly to ethyl ester 255-1 (710 mg, 2.92 mmol) in MeOH (29 mL) at 0° C. with stirring. The mixture was allowed to warm to room temperature. Consumption of the starting ethyl ester was observed by TLC (2% MeOH in DCM) after 5 hrs. The reaction mixture was concentrated under reduced pressure. The resulting oil was then cooled to 0° C. and 50 mL of 0.1 M HCl added. The pH aqueous phase was checked to ensure it was acidic. The aqueous phase was extracted three times with DCM, and the organics were combined, dried over anhydrous Na₂SO₄, filtered and concentrated to give a white powder. Over time, the water layer showed white suspension, thought to be additional product. Vacuum filtration was used to collect the suspension. The liquid filtrate was then extracted using ethyl acetate to increase yield further. Based on this second extraction of the filtrate, ethyl acetate seems to be a more efficient extraction solvent for this system than DCM. The original organic extract, the suspension collected from the water layer, and the organic layer collected from the extraction of the filtrate were combined to give the carboxylic acid 25 (255-2) as a white powder (88%) with no further purification. ¹H NMR (CDCl₃) was used to confirm the loss of the ethyl ester. ¹H NMR (500 MHz, CDCl₃): δ 5.77 (d, 1H, J³_{H-H}=7.1 Hz), 4.60 (ddd, 1H, J³_{H-H}=9.2 Hz, 7.8 Hz, 5.0 Hz), 2.12 (m, 3H) 1.78-1.67 (m, 2H), 1.59 (m, 1H), 0.96 (m, 12H).

### Example 15

This example demonstrates the synthesis of 3-Cyclohexyl-2-[4-methyl-2-(3-methyl-butyrylamino)-pentanoylamino]-propionic acid methyl ester (27, 255-3). To the solution of 25 (255-2) (423 mg, 1.96 mmol, 1 equiv) and D-cyclohexylalanine methyl ester hydrochloride (436 mg, 1.96 mmol, 1 equiv) in DCM (15 mL) was added diisopropylethylamine (DIEA) (0.92 mL, 5.3 mmol, 2.7 equiv) followed by HATU (1.49 g, 3.92 mmol, 2 equiv). The resulting mixture was stirred for overnight at room temperature. After 22 hrs, the TLC (5% MeOH in DCM) showed disappearance of the starting materials. The reaction mixture was quenched by washing with aqueous Na₂CO₃, followed by extraction with DCM. The organic layer was collected and washed with 0.01 M HCl and again extracted with DCM. The resulting organic layer was then washed with water, dried over anhydrous Na₂SO₄, filtered and concentrated. The crude orange solid (1.04 g) was purified through flash column chromatography (1% MeOH in DCM) to give the pure coupled product 27 (255-3) as a white solid (717 mg; 96%). ¹H NMR (500 MHz, CDCl₃): δ 6.57 (d, 1H, J³_{H-H}=8.1 Hz), 5.82 (d, 1H, J³_{H-H}=6.8 Hz), 4.49 (m, 2H), 3.63 (s, 3H), 2.03 (m, 3H), 1.80-1.52 (m, 9H), 1.46 (m, 2H), 1.29-0.99 (m, 5H), 0.88 (m, 12H). ¹³C NMR (500 MHz, CDCl₃): δ 173.1, 172.7, 171.8, 52.2, 51.3, 50.2, 45.9, 40.8, 39.8, 34.2, 33.5, 32.4, 26.3, 26.2, 26.0, 24.9, 22.8, 22.42, 22.37, 22.2. HRMS m/z calc for C₂₁H₃₈N₂O₄ (M+H)⁺ theory: 382.2832, found: 382.2820. Anal. Chem. C₂₁H₃₈N₂O₄, CHN.

### Example 16

This example demonstrates the synthesis of 4-Methyl-2-(3-methyl-butyrylamino)-pentanoic acid (1-carbamoyl-2-cyclohexyl-ethyl)-amide (28, 255-4). To a solution of 27 (255-3) (679.5 mg, 1.776 mmol, 1 equiv) in MeOH (20 mL) was added a vigorous stream of NH₃ gas at 0° C. with stirring. After 1 hr, the introduction of ammonia gas is discontinued and the flask closed with a glass stopper. The reaction solution was allowed to warm to room temperature and stirred for five days. After the first two days, a stream NH₃ gas was reintroduced for an additional hour. The solvent was removed under reduced pressure after 5 days to give the crude triamide product as a cream colored solid (670 mg). The crude solid was taken up in cold DCM (50 mL) and filtered to give the pure triamide 28 (255-4) as a white solid (534 mg, 82%). ¹H NMR (500 MHz, d₆-DMSO): δ 8.26 (d, 1H, J³_{H-H}=8.3 Hz), 8.02 (d, 1H, J³_{H-H}=6.6 Hz), 7.27 (s, 1H), 7.01 (s, 1H), 4.18 (m, 2H), 1.97 (m, 3H), 1.68-1.34 (m, 11H), 1.17-1.00 (m, 2H), 0.91 (m, 4H), 0.85 (m 11H). HRMS m/z calc for C₂₀H₃₇N₃O₃ (M+H)⁺ theory: 367.2835, found: 367.2871. Anal. Chem. C₂₀H₃₇N₃O₃, CHN.

### Example 17

This example demonstrates the synthesis of N1-(2-Amino-1-cyclohexylmethyl-ethyl)-4-methyl-N2-(3-methyl-butyl)-pentane-1,2-diamine (29, 255-5). Borane-tetrahydrofuran complex (1.0 M, 10.96 mmol, 10.96 mL, 8 equiv) was added via a syringe to N-[1-(1-Carbamoyl-2-phenyl-ethylcarbamoyl)-2-phenyl-ethyl]-3,3-dimethyl-butyramide 28 (255-4) (505 mg, 1.37 mmol, 1 equiv) in THF (15 mL) at ambient temperature. The reaction mixture was heated at 60-65° C. for 2 days before being concentrated under reduced pressure to give a residue. A 10% concentrated HCl/Methanol solution was then added at 0° C. and stirred for 24 hrs. The mixture was concentrated to give a white residue, which was taken up in 1 M NaOH until reaching pH 10, then extracted three times with DCM, dried over anhydrous Na₂SO₄, filtered, and concentrated to give triamine free base 29 (255-5) as an oil (97%) without further purification. ¹H NMR (500 MHz, CDCl₃): δ 2.71-2.39 (m, 7H), 2.31 (dd, 1H, J²_{H-H}=11.7 Hz, J³_{H-H}=6.8 Hz), 1.60 (m, 6H), 1.33-1.02 (m, 11H), 0.83 (dd, 1H, J³_{H-H}=6.6 Hz, J³_{H-H}=3.2 Hz). ¹³C NMR (500 MHz, CDCl₃): δ 56.9, 56.0, 50.2, 45.4, 45.0, 42.6, 40.7, 39.6, 34.6, 33.9, 33.7, 26.6, 26.4, 26.2, 25.1, 23.2, 22.9, 22.73, 22.70.

### Example 18

This example demonstrates the synthesis of N-{3-Cyclohexyl-2-[4-methyl-2-(3-methyl-butylamino)-pentylamino]-propyl}-benzamide (30, 255-6). A solution of N-(benzoyloxy)succinimide (259 mg, 1.18 mmol, 0.89 equiv) in DCM (2 mL) was added dropwise to a stirred solution of triamine 29 (255-5) (434 mg, 1.33 mmol, 1 equiv) in DCM (2 mL) at 0° C. The reaction mixture was allowed to warm to room temperature and stirred for 20 h. After TLC (7% MeOH, 1% NH₄OH in DCM) showed complete consumption of the starting material (20 hrs), the reaction mixture was washed with aqueous sodium carbonate, dried over anhydrous Na₂SO₄, filtered, and concentrated to give a crude residue. The crude was purified by flash column chromatography (2% MeOH, 1% NH₄OH in DCM) to give pure 30 (255-6) as an oil (73%). ¹H NMR (500 MHz, CDCl₃): δ 7.81 (m, 2H), 7.47 (m, 1H), 7.41 (m, 2H), 7.18 (br s, 1H), 3.57 (dt, 1H, J²_{H-H}=13.6 Hz, J³_{H-H}=4.4 Hz, 4.4 Hz), 3.27 (dt, 1H, J²_{H-H}=13.4 Hz, J³_{H-H}=5.6 Hz, 5.6 Hz), 2.84 (m, 1H), 2.79 (dd, 1H, J²_{H-H}=11.7 Hz, J³_{H-H}=3.7 Hz), 2.66 (m, 1H), 2.58 (m, 2H), 2.44 (dd, 1H, J²_{H-H}=11.7 Hz, J³_{H-H}=6.1 Hz), 1.70 (m, 4H), 1.60 (m, 2H), 1.44-1.10 (m, 11H), 0.88 (m, 14H). ¹³C NMR (500 MHz, CDCl₃): δ 167.4, 134.8, 131.2, 128.4, 127.0, 55.8, 54.0, 48.9, 45.3, 42.33, 42.26, 41.0, 39.4, 34.4, 33.6, 26.5, 26.3, 26.2, 25.1, 23.1, 22.70, 22.66, 22.6. HRMS m/z calc for C₂₇H₄₇N₃O (M+H)⁺ theory: 429.3719, found: 429.3719. Anal. Chem. C₂₇H₄₇N₃O.0.2H₂O, CHN.

### Example 19

This example demonstrates the synthesis of N-{3-Cyclohexyl-2-[5-isobutyl-4-(3-methyl-butyl)-2,3-dioxo-piperazin-1-yl]-propyl}-benzamide (31, 255-7). To a solution of diamine 30 (255-6) (0.02 M, 330 mg, 0.768 mmol, 1 equiv) in DCM (18 mL) at 0° C. was slowly added a 5-fold excess of oxalyldiimidazole (0.1 M, 730 mg, 3.84 mmol, 5 equiv) in DCM (20 mL). The resulting reaction mixture was allowed to stir at room temperature 3 days until complete consumption of the starting material was observed by TLC (7% MeOH, 1% NH₄OH in DCM). The mixture was then concentrated under reduced pressure. The crude reaction residue (1.099 g) was purified by flash column chromatography (2% MeOH in DCM) to give the pure cyclized product 31 (255-7) as a white powder (307 mg, 82%). ¹H NMR (500 MHz, CDCl₃): δ 7.75 (m, 2H), 7.45 (m, 1H), 7.38 (m, 2H), 7.00 (br s, 1H), 4.83 (m, 1H), 3.91 (m, 2H), 3.67 (dd, 1H, J²_{H-H}=13.0 Hz, J³_{H-H}=4.2 Hz), 3.40 (m 1H), 3.28 (dt, 1H, J²_{H-H}=14.1 Hz, J³_{H-H}=3.9 Hz, 3.9 Hz), 3.23 (dd, 1H, J²_{H-H}=13.2 Hz), 2.80 (m, 1H), 1.93 (d, 1H, J²_{H-H}=12.7 Hz), 1.69 (m, 5H), 1.59-1.31 (m, 7H), 1.30-1.09 (m, 4H), 0.92 (dd, 6H, J³_{H-H}=8.6 Hz, J³_{H-H}=6.6 Hz), 0.84 (dd, 6H, J³_{H-H}=8.6 Hz, J³_{H-H}=6.6 Hz). ¹³C NMR (500 MHz, CDCl₃): δ 168.0, 158.7, 156.3, 133.9, 131.5, 128.6, 127.1, 51.8, 44.3, 41.6, 40.1, 37.1, 36.6, 34.4, 34.0, 32.7, 26.4, 26.2, 26.1, 25.9, 25.2, 23.3, 22.6, 22.2, 21.2. HRMS m/z calc for C₂₉H₄₅N₃O₃ (M+H)⁺ theory: 483.3461, found: 483.3451. Anal. Chem. C₂₉H₄₅N₃O₃, CHN.

### Example 20

This example demonstrates the synthesis of Benzyl-{3-cyclohexyl-2-[3-isobutyl-4-(3-methyl-butyl)-piperazin-1-yl]-propyl}-amine trihydrochloride salt (10, 1666.255). Borane-tetrahydrofuran complex (1.0 M, 3.9 mL, 3.9 mmol, 8 equiv) was added via a syringe to 31 (255-7) (236 mg, 0.488 mmol, 1 equiv) in THF (6 mL) at ambient temperature. The mixture was then heated at 60-65° C. for 5 days. The reaction mixture was concentrated under reduced pressure to give a residue. A 10% concentrated HCl/Methanol solution (8 mL) was then added at 0° C. and stirred for 24 h. The mixture was concentrated to give a residue, which was taken up in 1 M NaOH until reaching pH 10, then extracted three times with DCM, dried over anhydrous Na₂SO₄, filtered, and concentrated. The crude triamine free base (176 mg) was purified by flash column chromatography (2% MeOH, 1% NH₄OH in DCM) to give the pure free base of 10 (1666.255) as a yellow oil (122 mg, 60%). Free base form of 10: ¹H NMR (500 MHz, CDCl₃): δ 7.31 (m, 3H), 7.24 (m, 2H), 3.79 (m, 2H), 2.80-2.57 (m, 5H), 2.55-2.43 (m, 3H), 2.34 (m, 3H), 2.13 (m, 1H), 1.76-1.47 (m, 7H), 1.44-1.07 (m, 9H), 0.89 (m, 12H). ¹³C NMR (500 MHz, CDCl₃): δ 140.5, 128.4, 126.8, 60.4, 58.1, 54.0, 51.7, 51.2, 49.7, 35.1, 34.32, 34.25, 33.1, 26.8, 26.6, 26.3, 26.2, 25.6, 24.1, 22.9, 22.7, 22.1. The free base of 10 (106 mg) was dissolved in absolute ethanol (6 mL) at 0° C. A 4 N HCl solution (12 mL) was slowly added to the free base solution. The solution was stirred for 30 minutes then concentrated. The resulting white solid was then taken up in water and concentrated to remove any remaining ethanol, giving the respective amine HCl salt of 10 (1666.255) (130.8 mg) as a crystalline solid. HRMS m/z calc for C₂₉H₅₁N₃ (M+H)⁺ theory: 441.4085, found: 441.4055.

### Example 21

### ³H-Leucine Uptake Assay

³H-Leucine uptake experiments were performed according to the protocol developed by Hälfliger et al. (2018) (referenced below with the following changes. Briefly, cells were seeded at 60% confluency in a 24-well plate and incubated for 4 h at 37° C. After 4 hours, different concentrations of compound 10 were added and the cells were then incubated overnight. L-³H-leucine uptake inhibition was measured for 15 minutes using a 12 μM stock of L-[³H]leucine (79 Ci/mmol). The final concentration of ³H-leucine in each well was 1.2 uM. This concentration produced a CPM reading of ˜4000 cpm for the control. Uptake was terminated by removing the buffer solution followed by washing the cells with cold Na⁺-free Hank's Balanced Salt Solution. Cells were then lysed to give ˜500 μL of cell lysate. A portion of the cell lysate (200 μL) was mixed with Scintiverse™ BD Cocktail for determining radiocounts. The radioactivity was measured with a scintillation counter (Beckman Coulter LS 6500 Multi-Purpose Scintillation Counter). Another portion of the cell lysate (300 μL) was used for protein determination using the BCA method. The final data was expressed as cpm/microgram of protein to account for the different number of cells remaining after the experiment. This was necessary because compound 10 was toxic and less cells were present after the overnight incubation (24 h). The results are shown in FIG. 10D (JPH-203, a known LAT-1 inhibitor) and 10E (compound 10).

### Example 22

³H-Leucine Efflux Assay ³H-Leucine efflux experiments were performed according to the protocol developed by Hälfliger et al. (2018) (referenced below) with the following changes. Briefly, cells were preloaded with 12 μM stock of L-[³H]leucine (79 Ci/mmol) to give a final concentration of ³H-leucine was 1.2 μM in each well. Cells were preloaded for 5 min at 37° C., then washed three times with cold Na⁺-free Hank's Balanced Salt Solution. Efflux was then induced by the presence or absence of the test compound (JPH-203 at 30 μM or compound 10 at 20 μM) for 2 minutes at 37° C. The medium was then collected and mixed with scintillation fluid (Scintiverse™ BD Cocktail) and radioactivity measured (Beckman Coulter LS 6500 Multi-Purpose Scintillation Counter). The cells were then washed three times with cold Na⁺-free Hank's Balanced Salt Solution, then lysed to give ˜300 μL of cell lysate. The lysate (300 μL) was mixed with scintillation fluid and radioactivity was counted. Relative efflux was expressed as percentage radioactivity=100%×(radioactivity of medium)/(radioactivity of the medium+radioactivity of the cells). The results are shown in FIG. 10F (JPH-203) and FIG. 10G (compound 10). This assay was repeated with a 30 min incubation time instead of two minutes and the results with compound 10 are shown in FIG. 10H.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C § 112, sixth paragraph. In particular, the use of “step of” in the claims herein is not intended to invoke the provisions of 35 U.S.C § 112, sixth paragraph.

All patents, patent applications, provisional applications, and publications referred to or cited herein, supra or infra, are incorporated by reference in their entirety, including all figures and tables, to the extent they are not inconsistent with the explicit teachings of this specification.

It should be emphasized that the above-described embodiments of the present disclosure are merely possible examples of implementations and are merely set forth for a clear understanding of the principles of this disclosure. It should be understood that the examples and embodiments described herein are for illustrative purposes only and that various modifications or changes in light thereof will be suggested to persons skilled in the art and are to be included within the spirit and purview of this application. Many variations and modifications may be made to the above-described embodiment(s) of the disclosure without departing substantially from the spirit and principles of the disclosure. All such modifications and variations are intended to be included herein within the scope of this disclosure and protected by the following claims.

### REFERENCES

1. Casero, R. A., Jr.; Marton, L. J., Targeting polyamine metabolism and function in cancer and other hyperproliferative diseases. Nature reviews. Drug discovery 2007, 6 (5), 373-390.
2. Igarashi, K.; Kashiwagi, K., Modulation of cellular function by polyamines. Int J Biochem Cell Biol 2010, 42 (1), 39-51.
3. Russell, D.; Snyder, S. H., Amine synthesis in rapidly growing tissues: ornithine decarboxylase activity in regenerating rat liver, chick embryo, and various tumors. Proc Natl Acad Sci USA 1968, 60 (4), 1420-1427.
4. Russell, D. H., The roles of the polyamines, putrescine, spermidine, and spermine in normal and malignant tissues. Life Sci 1973, 13 (12), 1635-1647.
5. Mandal, S.; Mandal, A.; Johansson, H. E.; Orjalo, A. V.; Park, M. H., Depletion of cellular polyamines, spermidine and spermine, causes a total arrest in translation and growth in mammalian cells. Proc Natl Acad Sci USA 2013, 110 (6), 2169-2174.
6. Kahana, C., Regulation of cellular polyamine levels and cellular proliferation by antizyme and antizyme inhibitor. Essays Biochem 2009, 46, 47-61.
7. Muth, A.; Madan, M.; Archer, J. J.; Ocampo, N.; Rodriguez, L.; Phanstiel, O., Polyamine transport inhibitors: design, synthesis, and combination therapies with difluoromethylornithine. J Med Chem 2014, 57 (2), 348-363.
8. Pegg, A. E., Mammalian polyamine metabolism and function. IUBMB Life 2009, 61 (9), 880-894.
9. Pegg, A. E., Polyamine metabolism and its importance in neoplastic growth and a target for chemotherapy. Cancer Res 1988, 48 (4), 759-774.
10. Massaro, C.; Thomas, J.; Phanstiel, O., Investigation of Polyamine Metabolism and Homeostasis in Pancreatic Cancers. Med Sci (Basel) 2017, 5 (4).
11. He, Y.; Shimogori, T.; Kashiwagi, K.; Shirahata, A.; Igarashi, K., Inhibition of cell growth by combination of alpha-difluoromethylornithine and an inhibitor of spermine synthase. J Biochem 1995, 117 (4), 824-829.
12. Gitto, S. B.; Pandey, V.; Oyer, J. L.; Copik, A. J.; Hogan, F. C.; Phanstiel, O.; Altomare, D. A., Difluoromethylornithine Combined with a Polyamine Transport Inhibitor Is Effective against Gemcitabine Resistant Pancreatic Cancer. Mol Pharm 2018, 15 (2), 369-376.
13. Kongpracha, P.; Nagamori, S.; Wiriyasermkul, P.; Tanaka, Y.; Kaneda, K.; Okuda, S.; Ohgaki, R.; Kanai, Y., Structure-activity relationship of a novel series of inhibitors for cancer type transporter L-type amino acid transporter 1 (LAT1). J Pharmacol Sci 2017, 133 (2), 96-102.
14. Nefzi, A.; Giulianotti, M. A.; Houghten, R. A., Solid-Phase Synthesis of Substituted 2,3-Diketopiperazines from Reduced Polyamides. Tetrahedron 2000, 56, 3319-3326.
15. Alhonen-Hongisto, L.; Seppanen, P.; Janne, J., Intracellular putrescine and spermidine deprivation induces increased uptake of the natural polyamines and methylglyoxal bis(guanylhydrazone). Biochem J 1980, 192 (3), 941-945.
16. Burns, M. R.; Carlson, C. L.; Vanderwerf, S. M.; Ziemer, J. R.; Weeks, R. S.; Cai, F.; Webb, H. K.; Graminski, G. F., Amino acid/spermine conjugates: polyamine amides as potent spermidine uptake inhibitors. J Med Chem 2001, 44 (22), 3632-3644.
17. Wang, C.; Delcros, J. G.; Biggerstaff, J.; Phanstiel, O., Synthesis and biological evaluation of N1-(anthracen-9-ylmethyl)triamines as molecular recognition elements for the polyamine transporter. J Med Chem 2003, 46 (13), 2663-2671.
18. Gardner, R. A.; Delcros, J. G.; Konate, F.; Breitbeil, F., 3rd; Martin, B.; Sigman, M.; Huang, M.; Phanstiel, O., N1-substituent effects in the selective delivery of polyamine conjugates into cells containing active polyamine transporters. J Med Chem 2004, 47 (24), 6055-6069.
19. Napolitano, L.; Galluccio, M.; Scalise, M.; Parravicini, C.; Palazzolo, L.; Eberini, I.; Indiveri, C., Novel insights into the transport mechanism of the human amino acid transporter LAT1 (SLC7A5). Probing critical residues for substrate translocation. Biochim Biophys Acta 2017, 1861 (4), 727-736.
20. Wright, P. S.; Byers, T. L.; Cross-Doersen, D. E.; McCann, P. P.; Bitonti, A. J., Irreversible inhibition of S-adenosylmethionine decarboxylase in Plasmodium falciparum-infected erythrocytes: growth inhibition in vitro. Biochemical pharmacology 1991, 41 (11), 1713-1718.
21. Pegg, A. E.; McCann, P. P., S-adenosylmethionine decarboxylase as an enzyme target for therapy. Pharmacol Ther 1992, 56 (3), 359-377.
22. Weeks, R. S.; Vanderwerf, S. M.; Carlson, C. L.; Burns, M. R.; O'Day, C. L.; Cai, F.; Devens, B. H.; Webb, H. K., Novel lysine-spermine conjugate inhibits polyamine transport and inhibits cell growth when given with DFMO. Exp Cell Res 2000, 261 (1), 293-302.
23. Yanagida, O.; Kanai, Y.; Chairoungdua, A.; Kim, D. K.; Segawa, H.; Nii, T.; Cha, S. H.; Matsuo, H.; Fukushima, J.; Fukasawa, Y.; Tani, Y.; Taketani, Y.; Uchino, H.; Kim, J. Y.; Inatomi, J.; Okayasu, I.; Miyamoto, K.; Takeda, E.; Goya, T.; Endou, H., Human L-type amino acid transporter 1 (LAT1): characterization of function and expression in tumor cell lines. Biochim Biophys Acta 2001, 1514 (2), 291-302.
24. Uemura, T.; Yerushalmi, H. F.; Tsaprailis, G.; Stringer, D. E.; Pastorian, K. E.; Hawel, L., 3rd; Byus, C. V.; Gerner, E. W., Identification and characterization of a diamine exporter in colon epithelial cells. The Journal of biological chemistry 2008, 283 (39), 26428-26435.
25. Driscoll, D. R.; Karim, S. A.; Sano, M.; Gay, D. M.; Jacob, W.; Yu, J.; Mizukami, Y.; Gopinathan, A.; Jodrell, D. I.; Evans, T. R.; Bardeesy, N.; Hall, M. N.; Quattrochi, B. J.; Klimstra, D. S.; Barry, S. T.; Sansom, O. J.; Lewis, B. C.; Morton, J. P., mTORC2 Signaling Drives the Development and Progression of Pancreatic Cancer. Cancer Res 2016, 76 (23), 6911-6923.
26. Dai, Z.-J.; Gao, J.; Ma, X.-B.; Kang, H.-F.; Wang, B.-F.; Lu, W.-F.; Lin, S. C.; Wang, X.-J.; Wu, W.-Y., Antitumor Effects of Rapamycin in Pancreatic Cancer Cells by Inducing Apoptosis and Autophagy. Int. J. Mol. Sci. 2013, 14, 273-285.
27. Mosmann, T., Rapid colorimetric assay for cellular growth and survival: application to proliferation and cytotoxicity assays. J Immunol Methods 1983, 65 (1-2), 55-63.
28. Minocha, S. C.; Minocha, R.; Robie, C. A., High-performance liquid chromatographic method for the determination of dansyl-polyamines. J. Chromatogr. 1990, 511, 177-183.
29. Effects of single amino acid deficiency on mRNA translation are markedly different for methionine versus leucine. Kevin M. Mazor, Leiming Dong, Yuanhui Mao, Robert V. Swanda, Shu-Bing Qian, Martha H. Stipanuk. Scientific Reports (2018) 8:8076; DOI:10.1038/s41598-018-26254-2.
30. Serum methionine depletion without side effects by methioninase in metastatic breast cancer patients. Tan Y1, Zavala J Sr, Xu M, Zavala J Jr, Hoffman R M. Anticancer Res. 1996 November-December; 16(6C):3937-42.
31. Hu J, Cheung N K. Methionine depletion with recombinant methioninase: in vitro and in vivo efficacy against neuroblastoma and its synergism with chemotherapeutic drugs. Int J Cancer. 2009; 124(7):1700-1706. doi:10.1002/ijc.24104.
32. Demetrius M. Kokkinakis, S. Clifford Schold Jr., Hiroki Hori & Tsutomu Nobori (1997) Effect of long-term depletion of plasma methionine on the growth and survival of human brain tumor xenografts in athymic mice, 29:3, 195-204, DOI: 10.1080/01635589709514624.
33. Designing the Polyamine Pharmacophore: Influence of N-substituents on the transport behavior of polyamine conjugates, Kaur, N.; Delcros, J-G.; Archer, J.; Weagraff, N. Z.; Martin, B.; Phanstiel I V, O. J. Med. Chem. 2008, 51, 2551-2560.
34. Robert M. Hoffman (ed.), Methionine Dependence of Cancer and Aging: Methods and Protocols, Methods in Molecular Biology, vol. 1866, Chapter 19 pp. 263-266. https://doi.org/10.1007/978-1-4939-8796-2_19).
35. C. Ye, B. M. Sutter, Y. Wang, Z. Kuang, B. P. Tu, A Metabolic Function for Phospholipid and Histone Methylation. Molecular Cell 2017, 66, 180-193.
36. Nicotinamide N-Methyltransferase: More Than a Vitamin B3 Clearance Enzyme. Trends in Endocrinology & Metabolism, 2017, 28, 340-353.
37. N. Zhang Role of methionine on epigenetic modification of DNA methylation and gene expression in animals. Anim. Nutr. 2018, 4, 11-16.
38. Samantha J. Mentch, Mahya Mehrmohamadi, Lei Huang, Xiaojing Liu, Diwakar Gupta, Dwight Mattocks, Paola Gómez Padilla, Gene Ables, Marcas M. Bamman, Anna E. Thalacker-Mercer, Sailendra N. Nichenametla, Jason W. Locasale. Histone Methylation Dynamics and Gene Regulation Occur through the Sensing of One-Carbon Metabolism. Cell Metabolism, 2015, 22, 861-873.
39. Häfliger P¹², Graff J³, Rubin M¹, Stooss A¹, Dettmer MS⁴, Altmann KH³, Gertsch J¹, Charles RP⁵. The LAT1 inhibitor JPH203 reduces growth of thyroid carcinoma in a fully immunocompetent mouse model. J Exp Clin Cancer Res. 2018, 37, 234. doi: 10.1186/s13046-018-0907-z.
40. Rohde, K. H, et al, Synthesis and antitubercular activity of 1,2,4-trisubstitued Piperazines Bioorganic & Medicinal Chemistry Letters 26 (2016) 2206-2209.
41. Yongye, A. B., et al, Identification, structure-activity relationships and molecular modeling of potent triamine and piperazine opioid ligands, Bioorganic & Medicinal Chemistry 17 (2009) 5583-5597.

What is claimed is:

1. A compound having a structure selected from Formula A, Formula B, and Formula C, 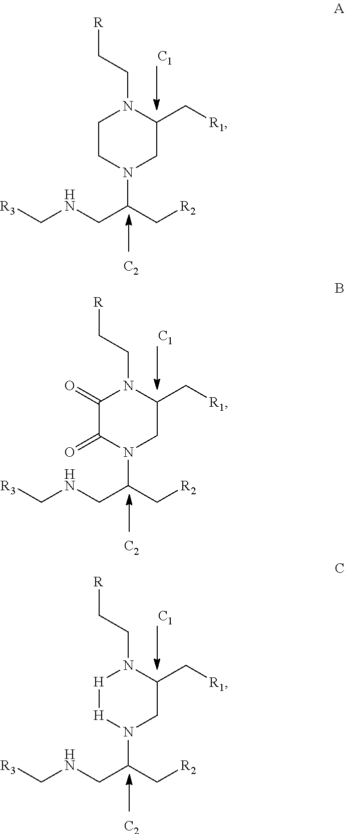
wherein R is selected from hydrogen, an aliphatic substituent, an alkylaryl substituent, a cycloalkyl substituent, an alkylcycloalkyl substituent and an aryl substituent,
wherein R₁ is selected from hydrogen, an aliphatic substituent, an alkylaryl substituent, a cycloalkyl substituent, an alkylcycloalkyl substituent, and an aryl substituent,
wherein R₂ is selected from hydrogen, an aliphatic substituent, an alkylaryl substituent, a cycloalkyl substituent, an alkylcycloalkyl substituent, and an aryl substituent,
wherein R₃ is selected from hydrogen, an aliphatic substituent, an alkylaryl substituent, a cycloalkyl substituent, an alkylcycloalkyl substituent, and an aryl substituent,
wherein C₁ is a first chiral center, C₂ is a second chiral center, and the compound has four stereoisomers, including an S,S stereoisomer, an R,R stereoisomer, an S,R stereoisomer, and an R,S stereoisomer.

2. The compound according to claim 1, wherein R is selected from methyl, ethyl, 1-propyl, 2-propyl, 1-butyl, isobutyl, sec-butyl, and tert-butyl.

3. The compound according to claim 1, wherein R is selected from cyclohexyl, phenyl, 4-fluorophenyl, benzyl, 4-fluorobenzyl, 2-pyridyl, and 3-pyridyl.

4. The compound according to claim 1, wherein R is selected from 1,1′-diphenylmethyl, or 3-(trifluoromethyl)phenyl, and bis-3,5-(trifluoromethyl)phenyl.

5. The compound according to claim 1, wherein R is selected from CH(CH₃)₂ and CH₂CH(CH₃)₂.

6. The compound according to claim 1, wherein R₁ is selected from 4-fluorophenyl, phenyl, 1-propyl, 2-propyl, isobutyl, sec-butyl, tert-butyl, 4-fluorobenzyl, and benzyl.

7. The compound according to claim 1, wherein R₁ is cyclohexyl.

8. The compound according to claim 1, wherein R₂ is hydrogen, methyl, ethyl, 1-propyl, 2-propyl, isobutyl, sec-butyl, tert-butyl, phenyl, benzyl, 4-hydroxyphenyl, 4-methoxyphenyl, 4-fluorophenyl, and cyclohexyl.

9. The compound according to claim 1, wherein R₃ is selected from hydrogen, cyclohexyl, 4-fluorophenyl, phenyl, 4-fluorobenzyl and benzyl.

10. The compound according to claim 1, wherein R₃ is selected from methyl, ethyl, 1-propyl, 2-propyl, butyl, sec-butyl, isobutyl, cyclohexyl and cyclohexylmethyl.

11. The compound according to claim 1, wherein R₃ is selected from cyclopentyl and 4-methylphenyl.

12. The compound according to claim 1, wherein R₃ is selected from 4-fluorophenyl, phenyl and cyclohexyl.

13. The compound according to claim 1, wherein the compound is the S,S stereoisomer.

14. The compound according to claim 1, wherein the compound is the R,R stereoisomer.

15. The compound according to claim 1, wherein the structure is Formula A, R is isopropyl, R₁ is isopropyl, R₂ is cyclohexyl, and R₃ is phenyl, where C₁ and C₂ are both in the S isomer configuration.

16. The compound according to claim 1, wherein the structure is Formula A, R is tert-butyl, R₁ is selected from phenyl or 4-fluorophenyl, R₂ is selected from cyclohexyl, phenyl or 4-fluorophenyl, and R₃ is selected from phenyl or 4-fluorophenyl, where C₁ and C₂ are both in the S isomer configuration.

17. The compound according to claim 1, wherein the structure is Formula A, R is isopropyl, R₁ is isopropyl, R₂ is cyclohexyl, and R₃ is selected from phenyl or 4-fluorophenyl, where C₁ and C₂ are both in the R isomer configuration.

18. The compound according to claim 1, wherein the structure is Formula A, R is t-butyl, R₁ is phenyl or 4-fluorophenyl, R₂ is selected from cyclohexyl, phenyl or 4-fluorophenyl, and R₃ is selected from phenyl or 4-fluorophenyl, where C₁ and C₂ are both in the R isomer configuration.

19. The compound according to claim 1, wherein the structure is Formula A, R is isopropyl, R₁ is isopropyl, R₂ is cyclohexyl, and R₃ is 4-fluorophenyl, where C₁ and C₂ are both in the S isomer configuration.

20. The compound according to claim 1, wherein the structure is Formula A, R is 2-propyl, R₁ is 2-propyl, R₂ is 2-propyl, and R₃ is 2-propyl, where C₁ and C₂ are both in the S isomer configuration:

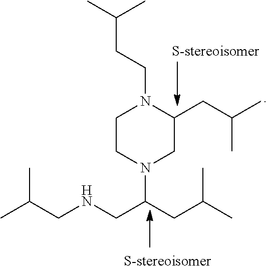

21. A method comprising administering an effective dosage of the compound according to claim 1 to a patient to treat a cancer.

22. The method according claim 21, wherein the cancer is selected from pancreatic cancer, breast cancer, colorectal cancer, prostate cancer, lung cancer, and melanoma.

23. A method comprising administering an effective dosage of the compound according to claim 1 to a patient to treat a parasitic disease, which relies on amino acid supply for survival.

24. The method according to claim 23, wherein the parasitic disease is selected from malaria, Leishmania, and Chagas disease.

25. A method comprising administering an effective dosage of the compound according to claim 1 to function as an intracellular depletion agent of one selected from leucine and methionine.

26. A method comprising administering an effective dosage of the compound according to claim 1 to function as a therapeutic in cells selected from mammalian cells and bacterial cells.

27. A therapeutic composition comprising the compound according to claim 1, and at least one antiproliferative agent.

28. The therapeutic composition according to claim 27, wherein the antiproliferative agent is selected from gemcitabine, difluoromethylornithine, a taxane derivative, and antifolate drugs.

29. The therapeutic composition according to claim 28, wherein the taxane derivative is taxol.

30. A method comprising administering an effective dosage of the compound according to claim 1 to function as a therapeutic which lowers intracellular methionine pools.

31. A method comprising administering an effective dosage of the compound according to claim 1 to a subject to function as a therapeutic which lowers intracellular methionine pools and to provide extended life span to the subject.

32. A method for synthesizing a compound having a structure selected from Formula A, Formula B, and Formula C, 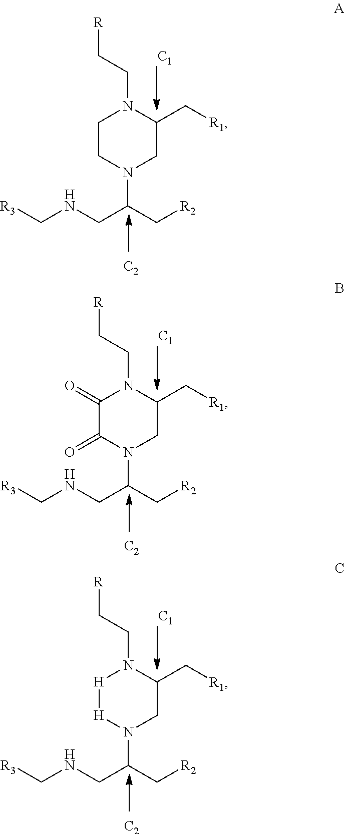
wherein R is selected from hydrogen, an aliphatic substituent, an alkylaryl substituent, a cycloalkyl substituent, an alkylcycloalkyl substituent and an aryl substituent
wherein R₁ is selected from hydrogen, an aliphatic substituent, an alkylaryl substituent, a cycloalkyl substituent, an alkylcycloalkyl substituent and an aryl substituent,
wherein R₂ is selected from hydrogen, an aliphatic substituent, an alkylaryl substituent, a cycloalkyl substituent, an alkylcycloalkyl substituent and an aryl substituent,
wherein R₃ is selected from hydrogen, an aliphatic substituent, an alkylaryl substituent, a cycloalkyl substituent, an alkylcycloalkyl substituent and an aryl substituent,
wherein C₁ is a first chiral center, C₂ is a second chiral center, and the compound has four stereoisomers, including an S,S stereoisomer, an R,R stereoisomer, an S,R stereoisomer, and an R,S stereoisomer,
the method comprising:
preparing a triamide scaffold;
preparing a chiral triamine by reducing the triamide scaffold;
preparing a diamine scaffold by regioselectively N-benzoylating the triamine scaffold; optionally regiospecifically cyclizing the diamine scaffold to prepare a cyclized scaffold; and reducing the diamine scaffold or the cyclized scaffold to form the compound.

33. The method according to claim 32, wherein preparing the triamide scaffold comprises coupling a plurality of peptides.

34. The method according to claim 32, wherein preparing the triamide scaffold comprises: coupling an N-acylated amino acid to either D- or L-cyclohexylalanine methyl ester hydrochloride to produce a diamidoester, and converting the diamidoester to the triamide scaffold using ammonia gas.
